(19) United States
(12) Patent Application Publication
(10) Pub. No.: US 2026/0124224 A1
(43) Pub. Date: May 7, 2026

(54) USE OF COMBINED COMPOSITION IN PREPARING DRUG FOR RESISTING FUNGAL AND BACTERIAL INFECTIONS

(71) Applicant: NATIONAL CENTER FOR NANOSCIENCE AND TECHNOLOGY, Beijing (CN)

(72) Inventors: Jing LIU, Beijing (CN); Mingge WANG, Beijing (CN); Chunying CHEN, Beijing (CN); Yuliang ZHAO, Beijing (CN)

(73) Assignee: NATIONAL CENTER FOR NANOSCIENCE AND TECHNOLOGY, Beijing (CN)

(21) Appl. No.: 19/318,683

(22) Filed: Sep. 4, 2025

(30) Foreign Application Priority Data
Nov. 7, 2024 (CN) 202411580607.1
Nov. 7, 2024 (CN) 202411582570.6

(51) Int. Cl. A61K 31/7048; A61K 9/1271; A61K 9/14; A61K 9/51; A61K 31/19; A61K 31/4196; A61P 31/04; A61P 31/10

(52) U.S. Cl. CPC A61K 31/7048; A61K 9/1271; A61K 9/14; A61K 9/5153; A61K 31/19; A61K 31/4196; A61P 31/04; A61P 31/10

(57) ABSTRACT

Provided is a use of a combined composition in preparing a drug for resisting fungal and bacterial infections. Active components of the combined composition are an antibiotic and an antibiotic adjuvant, wherein the antibiotic adjuvant is celastrol or a pharmaceutical salt thereof. The present application creatively discovers that celastrol or the pharmaceutical salt thereof can be combined with the antibiotic and the combination has a very excellent effect in resisting the fungal and bacterial infections, especially in resisting the fungal infections. Celastrol or the pharmaceutical salt thereof can enhance an antimicrobial effect of the antibiotic, especially an antifungal effect of the antibiotic, significantly increasing an antimicrobial sensitivity of the antibiotic and inhibiting the occurrence of fungal or bacterial drug-resistance. As an antibiotic adjuvant-antibiotic drug platform, the combined composition provides a new strategy for achieving an efficient antimicrobial effect, and the application dosage forms and the application scenarios are very diverse.

### CROSS-REFERENCE TO RELATED APPLICATION

This application claims priority to Chinese Patent Application No. 2024115825706 filed on Nov. 7, 2024, and Chinese Patent Application No. 2024115806071 filed on Nov. 7, 2024, the disclosures of which are incorporated herein by reference in their entirety.

### TECHNICAL FIELD

The present application belongs to the field of biomedicine and relates to a use of a combined composition in preparing a drug for resisting fungal and bacterial infections.

### BACKGROUND

Bacterial and fungal infections are important causes of human death, and the discovery of antibiotics significantly reduces the death of patients with bacterial and fungal infections. However, the abuse of antibiotics causes the rapid development of multidrug-resistant bacteria, posing a long-term threat to human health and sustainable production and development of grains. The development of new antibiotics is difficult and has a long period, seriously limiting the clinical treatment of drug-resistant bacterial infections. The arrival of the post-antibiotic era urges us to develop new treatment regimens to deal with the unoptimistic current situation of antibiotic resistance. The discovery of antibiotic adjuvants brings hope to deal with the current situations where the research and development of new antibiotics is hindered and existing drugs are difficult to deal with drug-resistant bacterial infections. Antibiotic adjuvants are used in combination with antibiotics to enhance the efficacy of antibiotics, providing new hope for restoring the activity of existing antibiotic drugs and inhibiting the generation of drug resistance, and providing an orthogonal strategy for the discovery of new antibiotics.

Tripterygium wilfordii, also known as Common Threewingnut Root, has multiple types of efficacies such as dispelling wind and dampness, promoting blood circulation and dredging collaterals, relieving swelling and pain, and reducing inflammation and detoxifying. Celastrol (CST), also known as tripterine, is a triterpenoid active ingredient in an extract of Tripterygium wilfordii and has multiple types of bioactivity. Studies have found that CST has effects such as anti-tumor, anti-rheumatoid arthritis and anti-Parkinson's disease. With the gradual deepening of research, various drug efficacies of CST have been continuously developed and applied. Nanotechnology is playing an increasingly important role in improving the bioavailability of drugs and reducing the toxic and side effect of drugs. The emergence of nanotechnology brings new strategies for improving the water solubility and bioavailability of hydrophobic drugs.

### SUMMARY

The following is a summary of the subject matter described in detail herein. This summary is not intended to limit the scope of the claims.

The present application is to provide a use of a combined composition in preparing a drug for resisting fungal and bacterial infections.

In a first aspect, the present application provides a use of a combined composition in preparing a drug for resisting fungal and bacterial infections, where active components of the combined composition are an antibiotic and an antibiotic adjuvant, and the antibiotic adjuvant is CST or a pharmaceutical salt thereof.

In a second aspect, the present application provides a method for treating or preventing fungal and bacterial infections, comprising administering an effective amount of a drug to subject in need thereof, wherein the drug comprises a combined composition;
wherein active components of the combined composition are an antibiotic and an antibiotic adjuvant, and the antibiotic adjuvant is CST or a pharmaceutical salt thereof.

The present application creatively discovers that CST or the pharmaceutical salt thereof can be combined with the antibiotic and the combination has a very excellent effect in resisting the fungal and bacterial infections, especially in resisting the fungal infections. CST or the pharmaceutical salt thereof can enhance an antimicrobial effect of the antibiotic, especially an antifungal effect of the antibiotic, increasing an antimicrobial sensitivity of the antibiotic and inhibiting the occurrence of fungal or bacterial drug-resistance. As an antibiotic adjuvant-antibiotic drug platform, the combined composition provides a new strategy for achieving an efficient antimicrobial effect, and the application dosage forms and the application scenarios are very diverse.

The above “pharmaceutical salt” includes an acidic salt or a basic salt.

The acidic salt includes hydrochloride, sulfate, phosphate, hydrobromide, acetate, benzoate, benzenesulfonate, tartrate, carbonate, citrate, gluconate, lactate, malate, mesylate, stearate, valerate or nitrate.

The basic salt includes a sodium salt, a calcium salt, a potassium salt, a zinc salt or a meglumine salt.

In an embodiment, the antibiotic includes any one or a combination of at least two of amphotericin B, nystatin, miconazole, ketoconazole, fluconazole, itraconazole, voriconazole, posaconazole, flucytosine, terbinafine, caspofungin, micafungin, anidulafungin, griseofulvin, penicillin, amoxicillin, ampicillin, cefradine, cefalexin, ceftazidime, imipenem, amikacin, gentamicin, doxycycline, tetracycline, minocycline, erythromycin, clarithromycin, azithromycin, sulfadiazine, ciprofloxacin, levofloxacin, metronidazole, clindamycin or fosfomycin.

In an embodiment, the fungi include any one or a combination of at least two of Candida albicans, Candida glabrata, Candida tropicalis, Candida krusei, Aspergillus fumigatus, Aspergillus niger, Aspergillus flavus, Aspergillus oryzae, Fusarium, Cryptococcus neoformans, Candida, Mucor, Histoplasma capsulatum, Scedosporium, Coccidioides, Cryptococcus gattii or Paracoccidioides.

In an embodiment, the bacteria include any one or a combination of at least two of Klebsiella pneumoniae, Acinetobacter baumannii, Pseudomonas aeruginosa, Escherichia coli (Enterinvasive Escherichia coli, Enteroaggregative Escherichia coli coli), Salmonella, Shigella, Streptococcus pneumoniae, Staphylococcus aureus, Mycobacterium tuberculosis or Clostridioides difficile.

In an embodiment, a molar ratio of the antibiotic to the antibiotic adjuvant is 1:(0.001-500), for example, 1:0.001, 1:0.01, 1:0.05, 1:0.1, 1:0.5, 1:1, 1:5, 1:20, 1:30, 1:40, 1:50, 1:60, 1:70, 1:80, 1:100, 1:150, 1:200, 1:300, 1:400 or 1:500. Other specific point values within the numerical range may be selected, and details are not described here.

In an embodiment, the combined composition is a single compound preparation, a combination of an antibiotic adjuvant and an antibiotic each in separate preparations, or a nanoparticle.

In an embodiment, when the combined composition is the combination of the antibiotic adjuvant and the antibiotic each in separate preparations, a mode of administration of the combination of the antibiotic adjuvant and the antibiotic each in separate preparations may be simultaneous administration, cross administration or sequential administration.

In the present application, CST or the pharmaceutical salt thereof and the antibiotic are prepared into nanoparticles through nanotechnology, effectively improving the bioavailability of the antibiotic and improving the biocompatibility of CST or the pharmaceutical salt thereof. Moreover, CST or the pharmaceutical salt thereof has excellent effects in resisting fungal and bacterial infections. CST or the pharmaceutical salt thereof can enhance an antimicrobial effect of the antibiotic, especially an antifungal effect of the antibiotic, increasing an antimicrobial sensitivity of the antibiotic and inhibiting the occurrence of fungal or bacterial drug-resistance.

In an embodiment, when the combined composition is the nanoparticle, the combined composition includes the following three forms:
in form 1, the combined composition includes carrier-free nanoparticles formed by an antibiotic and an antibiotic adjuvant;
in form 2, the combined composition includes a composite liposome prepared from an antibiotic, an antibiotic adjuvant, a lipid and cholesterol that are used as raw materials;
in an embodiment, the lipid includes any one or a combination of at least two of phosphatidylcholine, phosphatidylethanolamine, phosphatidylinositol, phosphatidic acid, cardiolipin, sphingomyelin, phosphatidylserine, DSPE-PEG or fluorine-modified DSPE-PEG;
in form 3, the combined composition includes composite polymer nanoparticles prepared from an antibiotic, an antibiotic adjuvant and a pharmaceutical degradable polymer that are used as raw materials;
in an embodiment, the pharmaceutical degradable polymer includes any one or a combination of at least two of PLGA, PEG, mPEG-PLGA or DSPE-PEG;
in an embodiment, the preparation is any one of the pharmaceutically acceptable dosage forms.

In an embodiment, a dosage form of the preparation includes an oral dosage form, an injection dosage form or a topical dosage form.

In an embodiment, a mode of administration of the drug includes digestive tract administration, injection administration, transdermal administration, mucosal administration, inhalation administration or topical administration.

In an embodiment, the combined composition further includes a pharmaceutically acceptable adjuvant.

In an embodiment, the adjuvant includes any one or a combination of at least two of a carrier, a diluent, an adhesive, a wetting agent, a disintegrant, an emulsifier, a co-solvent, a solubilizer, an osmotic pressure regulator, a surfactant, a coating material, a colorant, a pH adjusting agent, an antioxidant, a bacteriostatic agent or a buffering agent.

In an embodiment, the carrier-free nanoparticles are prepared through a preparation method including the following steps:
(1) dissolving CST or a pharmaceutical salt thereof and a hydrophobic antibiotic in a good solvent to obtain a solution A;
(2) in a stirred environment, adding the solution A to a poor solvent of CST or the pharmaceutical salt thereof and the hydrophobic antibiotic to obtain a solution B; and
(3) separating free drugs to obtain the carrier-free nanoparticles.

In an embodiment, the good solvent includes any one or a combination of at least two of methanol, ethanol, propanol, isopropanol, acetone, tetrahydrofuran or dimethylsulfoxide.

In an embodiment, the poor solvent includes deionized water and/or a phosphate buffer.

In an embodiment, the stirring is performed for 5-60 min, for example, 5 min, 10 min, 20 min, 25 min, 30 min, 35 min, 40 min, 45 min, 50 min, 55 min or 60 min. Other specific point values within the numerical range may be selected, and details are not described here.

In an embodiment, ultrafiltration centrifugation is used in the separation of the free drugs, and conditions of the ultrafiltration centrifugation are a rotational speed of 8000-10000 g (for example, 8000 g, 8500 g, 9000 g, 9500 g or 10000 g) and a duration of 10-30 min (for example, 10 min, 15 min, 20 min, 25 min or 30 min). Other specific point values within the numerical ranges may be selected, and details are not described here.

In an embodiment, the composite liposome is prepared through a preparation method including the following steps:
(1) mixing a first solvent, CST or a pharmaceutical salt thereof, a hydrophobic antibiotic, a phospholipid and cholesterol to obtain a solution A, and mixing a second solvent with a hydrophilic antibiotic to obtain a solution B;
(2) removing the first solvent in the solution A, mixing the solute with the solution B, and hydrating the solute to obtain a solution C; and
(3) performing ultrasonication and gradient extrusion on the solution C to obtain the composite liposome.

In an embodiment, the first solvent includes any one or a combination of at least two of methanol, ethanol, propanol or chloroform, and the second solvent includes deionized water and/or a phosphate buffer.

In an embodiment, the hydration is performed for 10-60 min (for example, 10 min, 15 min, 20 min, 25 min, 30 min, 35 min, 40 min, 45 min, 50 min, 55 min or 60 min) at a temperature of 15-35° C. (for example, 15° C., 18° C., 20° C., 22° C., 25° C., 28° C., 30° C. or 35° C.). Other specific point values within the numerical ranges may be selected, and details are not described here.

In an embodiment, apertures of filter membranes for the gradient extrusion are 400 nm and 200 nm in sequence.

In an embodiment, the ultrasonic treatment in step (3) is performed at 0-10° C. (for example, 1° C., 2° C., 3° C., 4° C., 5° C., 7° C., 8° C. or 10° C.), and the ultrasonication is performed at a power of 80-250 W (for example, 80 W, 100 W, 120 W, 150 W, 180 W, 200 W, 220 W or 250 W) for 3-30 min (for example, 4 min, 8 min, 10 min, 12 min, 15 min, 20 min, 25 min or 30 min). Other specific point values within the numerical ranges may be selected, and details are not described here.

In an embodiment, the composite liposome is prepared through a preparation method including the following steps:
(1) mixing a first solvent, a phospholipid and cholesterol to obtain a solution A, mixing CST or a pharmaceutical salt thereof, an antibiotic and an organic solvent, and adding the mixture dropwise to a second solvent to obtain a solution B;
(2) removing the first solvent in the solution A, mixing the solute with the solution B, and hydrating the solute to obtain a solution C; and
(3) performing ultrasonication and gradient extrusion on the solution C to obtain the composite liposome.

In an embodiment, the first solvent includes any one or a combination of at least two of methanol, ethanol, propanol or chloroform, the organic solvent includes any one or a combination of at least two of methanol, ethanol, dimethylformamide (DMF) or dimethyl sulfoxide (DMSO), and the second solvent includes deionized water and/or a phosphate buffer.

In an embodiment, the hydration is performed for 10-30 min (for example, 10 min, 15 min, 20 min, 25 min or 30 min) at a temperature of 15-35° C. (for example, 15° C., 18° C., 20° C., 22° C., 25° C., 28° C., 30° C. or 35° C.). Other specific point values within the numerical ranges may be selected, and details are not described here.

In an embodiment, the ultrasonic treatment in step (3) is performed at 0-10° C. (for example, 1° C., 2° C., 3° C., 4° C., 5° C., 7° C., 8° C. or 10° C.), and the ultrasonication is performed at a power of 80-250 W (for example, 80 W, 100 W, 120 W, 150 W, 180 W, 200 W, 220 W or 250 W) for 3-30 min (for example, 4 min, 8 min, 10 min, 12 min, 15 min, 20 min, 25 min or 30 min). Other specific point values within the numerical ranges may be selected, and details are not described here.

In an embodiment, apertures of filter membranes for the gradient extrusion are 400 nm and 200 nm in sequence.

In an embodiment, the composite polymer nanoparticles are prepared through a preparation method including the following steps:
(1) mixing a first solvent with a pharmaceutical degradable polymer to obtain a solution A, mixing CST or a pharmaceutical salt thereof, an antibiotic and an organic solvent, and adding the mixture dropwise to a second solvent containing an emulsifier to obtain a solution B;
(2) mixing the solution A with the solution B to obtain a solution C;
(3) adding the solution C to a third solvent containing an emulsifier, and mixing to obtain a solution D; and
(4) removing the first solvent remaining in the solution D to obtain the composite polymer nanoparticles.

In an embodiment, the first solvent includes any one or a combination of at least two of methanol, ethanol, propanol or dichloromethane, the organic solvent includes any one or a combination of at least two of methanol, ethanol, DMF or DMSO, the second solvent includes deionized water and/or a phosphate buffer, and the third solvent includes deionized water and/or a phosphate buffer.

In an embodiment, the emulsifiers in the second solvent and the third solvent each independently include any one or a combination of at least two of polyvinyl alcohol, polysorbate 80 or vitamin E polyethylene glycol succinate.

In an embodiment, the emulsifier in the second solvent has a mass fraction of 3%-15% (for example, 3%, 7%, 8%, 10%, 12%, 14% or 15%), and the emulsifier in the third solvent has a mass fraction of 30% to 60% (for example, 30%, 35%, 40%, 45%, 50%, 55% or 60%).

In a third aspect, the present application provides a combined pharmaceutical composition for resisting fungal and bacterial infections, where active components of the combined pharmaceutical composition are an antibiotic and an antibiotic adjuvant.

The antibiotic adjuvant is CST or a pharmaceutical salt thereof.

The antibiotic includes any one or a combination of at least two of amphotericin B, nystatin, miconazole, ketoconazole, fluconazole, itraconazole, voriconazole, posaconazole, flucytosine, terbinafine, caspofungin, micafungin, anidulafungin, griseofulvin, penicillin, amoxicillin, ampicillin, cefradine, cefalexin, ceftazidime, imipenem, amikacin, gentamicin, doxycycline, tetracycline, minocycline, erythromycin, clarithromycin, azithromycin, sulfadiazine, ciprofloxacin, levofloxacin, metronidazole, clindamycin or fosfomycin.

In the present application, CST or the pharmaceutical salt thereof is used as the antibiotic adjuvant, and the antibiotic adjuvant is used in combination with multiple types of antibiotics to construct an antibiotic adjuvant-antibiotic drug platform; with the help of an antibiotic adjuvant effect of CST, the antimicrobial activity of the antibiotic is improved, especially the antifungal activity, and the occurrence of the drug resistance of bacteria and fungi is reduced. Compared with the treatment with the antibiotic alone, the antibiotic adjuvant-antibiotic drug platform reduces the minimum effective therapeutic concentration of the antibiotic, effectively broadens the therapeutic window of the drug and improves the safety of the treatment. Moreover, the antibiotic adjuvant-antibiotic drug platform can be prepared into various dosage forms to be applied to different treatment scenarios and meet requirements of different antibiotics for physical and chemical characteristics. Moreover, a clinical oral, injection, inhalation or smearing administration requirement can be met.

The above “pharmaceutical salt” includes an acidic salt or a basic salt.

The acidic salt includes hydrochloride, sulfate, phosphate, hydrobromide, acetate, benzoate, benzenesulfonate, tartrate, carbonate, citrate, gluconate, lactate, malate, mesylate, stearate, valerate or nitrate.

The basic salt includes a sodium salt, a calcium salt, a potassium salt, a zinc salt or a meglumine salt.

In an embodiment, a molar ratio of the antibiotic to the antibiotic adjuvant is 1:(0.001-500), for example, 1:0.001, 1:0.01, 1:0.05, 1:0.1, 1:0.5, 1:1, 1:5, 1:20, 1:30, 1:40, 1:50, 1:60, 1:70, 1:80, 1:100, 1:150, 1:200, 1:300, 1:400 or 1:500. Other specific point values within the numerical range may be selected, and details are not described here.

In an embodiment, the fungi include any one or a combination of at least two of Candida albicans, Candida glabrata, Candida tropicalis, Candida krusei, Aspergillus fumigatus, Aspergillus niger, Aspergillus flavus, Aspergillus oryzae, Fusarium, Cryptococcus neoformans, Candida, Mucor, Histoplasma capsulatum, Scedosporium, Coccidioides, Cryptococcus gattii or Paracoccidioides.

In an embodiment, the bacteria include any one or a combination of at least two of Klebsiella pneumoniae, Acinetobacter baumannii, Pseudomonas aeruginosa, Escherichia coli (Enterinvasive Escherichia coli, Enteroaggregative Escherichia coli coli), Salmonella, Shigella, Streptococcus pneumoniae, Staphylococcus aureus, Mycobacterium tuberculosis or Clostridioides difficile.

Compared with the related art, the present application has the beneficial effects below.

The present application creatively discovers that CST or the pharmaceutical salt thereof can be combined with the antibiotic and the combination has a very excellent effect in resisting the fungal and bacterial infections, especially in resisting the fungal infections. CST or the pharmaceutical salt thereof can enhance an antimicrobial effect of the antibiotic, especially an antifungal effect of the antibiotic, increasing an antimicrobial sensitivity of the antibiotic and inhibiting the occurrence of fungal or bacterial drug-resistance.

In the present application, CST or the pharmaceutical salt thereof is used as the antibiotic adjuvant, and the antibiotic adjuvant is used in combination with multiple types of antibiotics to construct an antibiotic adjuvant-antibiotic drug platform; with the help of an antibiotic adjuvant effect of CST, the antimicrobial activity of the antibiotic is improved, especially the antifungal activity, and the occurrence of the drug resistance of bacteria and fungi is reduced. Compared with the treatment with the antibiotic alone, the antibiotic adjuvant-antibiotic drug platform reduces the minimum effective therapeutic concentration of the antibiotic, effectively broadens the therapeutic window of the drug and improves the safety of the treatment.

After the antibiotic and CST are prepared into the nanoparticles, the antimicrobial sensitivity of the antibiotic can be further improved, inhibiting the occurrence of the drug resistance of fungi or bacteria. When the nanoparticles exist in a form of composite liposomes, the antibiotic and CST have a better effect in resisting the fungal and bacterial infections.

Moreover, the introduction of too many toxic organic reagents and carriers is avoided in a preparation process of the nanoparticles involved in the present application, thereby preventing the possible problem of biotoxicity. In addition, with simple and mild conditions, the preparation method is green, environmentally friendly and low-cost with no need for a special reaction device, which is easy to popularize and apply.

The antibiotic adjuvant-antibiotic drug platform can be prepared into various dosage forms to be applied to different treatment scenarios and meet requirements of different antibiotics for physical and chemical characteristics. Moreover, a clinical oral, injection, inhalation or smearing administration requirement can be met.

Other aspects can be understood after the drawings and the detailed description are read and understood.

### BRIEF DESCRIPTION OF DRAWINGS

The drawings are used to provide a further understanding of technical solutions herein, constitute part of the specification, and explain the technical solutions herein in conjunction with examples of the present application and are not intended to limit the technical solutions herein.

FIG. 1 is a diagram illustrating fractional inhibitory concentration index (FICI) results of a combined bacteriostatic experiment of CST and multiple types of antibiotics.

FIG. 2 is a diagram illustrating FICI results of a combined bacteriostatic experiment of CST and multiple types of antibiotics.

FIG. 3 is a diagram illustrating molecular docking results of CST and CYP51.

FIG. 4 is a diagram illustrating antifungal kinetics curves of a CST-Flu combined composition against Fusarium and Aspergillus fumigatus.

FIG. 5 is a diagram illustrating an antifungal kinetics curve of a composition of CST or a pharmaceutical salt thereof in combination with fluconazole against Aspergillus fumigatus.

FIG. 6 is a diagram illustrating detection results of hydrated particle sizes of carrier-free nanoparticles prepared in Preparation Example 1.

FIG. 7 is a diagram illustrating detection results of potentials of carrier-free nanoparticles prepared in Preparation Example 1.

FIG. 8 is a diagram illustrating detection results of hydrated particle sizes of carrier-free nanoparticles prepared in Preparation Example 2 in a phosphate-buffered saline (PBS) solution containing 10% fetal bovine serum (FBS) or ultrapure water at different times.

FIG. 9 is a diagram illustrating detection results of potentials of carrier-free nanoparticles prepared in Preparation Example 2 in a PBS solution containing 10% FBS or ultrapure water at different times.

FIG. 10 is a diagram illustrating an in vitro release curve of carrier-free nanoparticles prepared in Preparation Example 2 in a neutral or slightly acidic environment.

FIG. 11 is a diagram illustrating results of effects of a composite liposome (Preparation Example 9), CST and fluconazole (Flu) on cell membranes of Aspergillus fumigatus and drug-resistant strains thereof verified through a crystal violet biofilm staining experiment.

FIG. 12 is a diagram illustrating statistical results of hemolysis rates of carrier-free nanoparticles prepared in Preparation Examples 3 and 4.

FIG. 13 is a diagram illustrating detection results of a hydrated particle size of a composite liposome prepared in Preparation Example 5.

FIG. 14 is a diagram illustrating detection results of a potential of a composite liposome prepared in Preparation Example 5.

FIG. 15 is a diagram illustrating detection results of hydrated particle sizes of a composite liposome prepared in Preparation Example 7 in a PBS solution containing 10% FBS or ultrapure water at different times.

FIG. 16 is a diagram illustrating detection results of potentials of a composite liposome prepared in Preparation Example 7 in a PBS solution containing 10% FBS or ultrapure water at different times.

FIG. 17 is a diagram illustrating an in vitro release curve of a composite liposome prepared in Preparation Example 7 in a neutral or slightly acidic environment.

FIG. 18 is a diagram illustrating survival curves of Galleria mellonella larvae with fungal infections treated by using a composite liposome prepared in Preparation Example 9.

### DETAILED DESCRIPTION

Embodiments are provided hereinafter to illustrate technical solutions of the present application. It is to be understood by those skilled in the art that the examples are intended to facilitate understanding of the present application and are not to be construed as limiting the present application.

### Example 1

### Evaluation of Minimum Inhibitory Concentrations (MICs) of Drugs Alone

In this example, MICs of CST alone and various antibiotics alone (including amphotericin B-AmB, nystatin-Nys, miconazole-Mnc, ketoconazole-Ket, fluconazole-Flu, itraconazole-Itr, voriconazole-Vor, posaconazole-Pos, flucytosine-Fly, terbinafine-Ter, caspofungin-Cas, micafungin-Mcf, anidulafungin-Anf, griseofulvin-Gri, penicillin-Ben, amoxicillin-Amo, ampicillin-Amp, cefradine-Cfd, cefalexin-Cfa, ceftazidime-Cef, imipenem-Imi, amikacin-Ami, gentamicin-Gen, doxycycline-Dox, tetracycline-Tet, minocycline-Min, erythromycin-Ery, clarithromycin-Cla, azithromycin-Azi, sulfadiazine-Sul, ciprofloxacin-Cip, levofloxacin-Lev, metronidazole-Met, clindamycin-Cli or fosfomycin-Fos) against various types of fungi (including Candida albicans, Candida glabrata, Candida tropicalis, Candida krusei, Aspergillus fumigatus, Aspergillus niger, Aspergillus flavus, Aspergillus oryzae, Fusarium, Cryptococcus neoformans, Candida, Mucor, Histoplasma capsulatum, Scedosporium, Coccidioides, Cryptococcus gattii or Paracoccidioides) and bacteria (including Klebsiella pneumoniae, Acinetobacter baumannii, Pseudomonas aeruginosa, Enterinvasive Escherichia coli, Enteroaggregative Escherichia coli, Salmonella, Shigella, Streptococcus pneumoniae, Staphylococcus aureus, Mycobacterium tuberculosis or Clostridioides difficile) were measured. Specific operations are described below.
(1) The to-be-measured fungi or bacteria were inoculated on various types of solid culture plates (see Table 1 for details) and cultured in an incubator for 24 h at 37° C.
(2) Monoclonal strains in the solid media were picked, inoculated in various types of liquid media (see Table 1 for details) and cultured in the incubator for 24 h at 37° C.
(3) An appropriate amount of to-be-measured antibiotic drug powder or CST was weighed. After sterilized double-distilled water was added for thorough dissolution, the mixture was filtered through a 0.22 μm filter membrane to be prepared into a stock solution (0.64 mg/mL) for later use.
(4) The above stock solution was diluted to the highest to-be-measured drug concentration (64 μg/mL) by using a liquid medium, and a sterile 96-well plate was taken for sequentially performing double dilution in a biological safety cabinet:

200 μL of the drug with the highest to-be-measured concentration was added to the first well (A1), and 100 μL of liquid medium was added to each of the wells A2 to A12, 100 μL was aspirated from the well A1 and added to the well A2; after thorough mixing, 100 μL was aspirated from the well A2 and added to the well A3, and so on; gradient dilution was performed until the well A12 to ensure that each well finally contains 100 μL of the liquid medium containing the drug, and three parallel groups were repeated for each well.
(5) 1 mL of liquid medium was added to a clear plastic test tube, and the clear plastic test tube was placed on a turbidimeter for zero adjustment. Subsequently, the liquid medium containing the above freshly cultured to-be-measured strain was aspirated and blown uniformly, the turbidity was adjusted to 0.5 McFarland (MCF) turbidity, and the liquid medium containing the above freshly cultured to-be-measured strain was diluted 20 times by using a liquid medium for later use.
(6) 10 μL of the diluted bacterial suspension in each group was taken and sequentially added to the drug-containing liquid medium prepared in step (4) in each well, OD₆₀₀ in each well was detected by using a microplate reader, and the 96-well plate was placed in the incubator at 37° C. and cultured for 16 h. OD₆₀₀ in each well was monitored by using the microplate reader. The lowest drug concentration without bacterial growth (there is no significant change in OD₆₀₀ value during the detection) is the MIC of the drug against the bacteria.

The results are shown in Table 2 to Table 8.

TABLE 1

| Fungi/Bacteria | Solid Culture Plate | Liquid Medium |
|---|---|---|
| Candida albicans | SDA medium | SDB medium |
| Candida glabrata | SDA medium | SDB medium |
| Candida tropicalis | SDA medium | SDB medium |
| Candida krusei | SDA medium | SDB medium |
| Aspergillus fumigatus | SDA medium | SDB medium |
| Aspergillus niger | PDA medium | PDB medium |
| Aspergillus flavus | SDA medium | SDB medium |
| Aspergillus oryzae | PDA medium | PDB medium |
| Fusarium | PDA medium | PDB medium |
| Cryptococcus | SDA medium | SDB medium |
| neoformans | | |
| Candida | YPD agar medium | YPD medium |
| Mucor | blood agar medium | mildew liquid |
| | | medium-021070 |
| Histoplasma capsulatum | SDA medium | SDB medium |
| culture of Madurella | SDA medium | SDB medium |
| mycetomatis | | |
| Scedosporium | PDA medium | PDB medium |
| Coccidioides | blood agar | SDB medium |
| Cryptococcus gattii | CGB medium | SDB medium |
| Paracoccidioides | SDA medium | SDB medium |
| Klebsiella pneumoniae | blood agar plate | LB medium |
| Acinetobacter baumannii | blood agar plate | LB medium |
| Pseudomonas aeruginosa | beef extract-peptone | LB medium |
| | agar medium | |
| Escherichia coli | beef extract-peptone | LB medium |
| | agar medium | |
| Salmonella | BS agar plate | TTB medium |
| Shigella | beef extract-peptone | LB medium |
| | agar medium | |
| Streptococcus | blood agar medium | BHI medium |
| pneumoniae | | |
| Staphylococcus aureus | NA medium | BHI medium |
| Mycobacterium | Löwenstein-Jensen | Sauton's medium |
| tuberculosis | solid medium | |
| Clostridioides difficile | blood agar medium | cooked meat medium |

### Example 2

### Evaluation of MICs of Combined Compositions

In this example, MICs of CST and various antibiotics (including amphotericin B-AmB, nystatin-Nys, miconazole-Mnc, ketoconazole-Ket, fluconazole-Flu, itraconazole-Itr, voriconazole-Vor, posaconazole-Pos, flucytosine-Fly, terbinafine-Ter, caspofungin-Cas, micafungin-Mcf, anidulafungin-Anf, griseofulvin-Gri, penicillin-Ben, amoxicillin-Amo, ampicillin-Amp, cefradine-Cfd, cefalexin-Cfa, ceftazidime-Cef, imipenem-Imi, amikacin-Ami, gentamicin-Gen, doxycycline-Dox, tetracycline-Tet, minocycline-Min, erythromycin-Ery, clarithromycin-Cla, azithromycin-Azi, sulfadiazine-Sul, ciprofloxacin-Cip, levofloxacin-Lev, metronidazole-Met, clindamycin-Cli or fosfomycin-Fos) against various types of fungi (including Candida albicans, Candida glabrata, Candida tropicalis, Candida krusei, Aspergillus fumigatus, Aspergillus niger, Aspergillus flavus, Aspergillus oryzae, Fusarium, Cryptococcus neoformans, Candida, Mucor, Histoplasma capsulatum, Scedosporium, Coccidioides, Cryptococcus gattii or Paracoccidioides) and bacteria (including Klebsiella pneumoniae, Acinetobacter baumannii, Pseudomonas aeruginosa, Enterinvasive Escherichia coli, Enteroaggregative Escherichia coli, Salmonella, Shigella, Streptococcus pneumoniae, Staphylococcus aureus, Mycobacterium tuberculosis or Clostridioides difficile) were measured. Specific operations are described below.
(1) The to-be-measured fungi or bacteria were inoculated on various types of solid culture plates (see Table 1 for details) and cultured in an incubator for 24 h at 37° C.
(2) Monoclonal strains in the solid media were picked, inoculated in various types of liquid media (see Table 1 for details) and cultured in the incubator for 24 h at 37° C.
(3) An appropriate amount of to-be-measured antibiotic drug powder or CST was weighed. After sterilized double-distilled water was added for thorough dissolution, the mixture was filtered through a 0.22 μm filter membrane to be prepared into a stock solution (0.64 mg/mL) for later use.
(4) Gradient stock solutions were prepared according to the gradient dilution method in Example 1 (128 μg/mL, 64 μg/mL, 32 μg/mL, 16 μg/mL, 8 μg/mL, 4 μg/mL, 2 μg/mL, 1 μg/mL, 0.5 μg/mL, 0.25 μg/mL, 0.125 μg/mL and 0.0625 μg/mL, and 0.6 mL for each concentration) for later use.
(5) A sterile 96-well plate was taken for performing gradient preparation of the compositions in a biological safety cabinet:
   the first group: 50 μL of the 128 μg/mL antibiotic was added to each of the wells A1 to A12, 50 μL of each of the CST with a concentration of 128 μg/mL, 64 μg/mL, 32 μg/mL, 16 μg/mL, 8 μg/mL, 4 μg/mL, 2 μg/mL, 1 μg/mL, 0.5 μg/mL, 0.25 μg/mL, 0.125 μg/mL and 0.0625 μg/mL was added to the wells A1 to A12 in sequence, and three parallel groups were repeated for each well;
   the second group: 50 μL of the 64 μg/mL antibiotic was added to each of the wells B1 to B12, 50 μL of each of the CST with concentration gradients shown in the first group was added to the wells B1 to B12 in sequence, and three parallel groups were repeated for each well;
   the third group: 50 μL of the 32 μg/mL antibiotic was added to each of the wells C1 to C12, 50 μL of each of the CST with concentration gradients shown in the first group was added to the wells C1 to C12 in sequence, and three parallel groups were repeated for each well;
   the fourth group: 50 μL of the 16 μg/mL antibiotic was added to each of the wells D1 to D12, 50 μL of each of the CST with concentration gradients shown in the first group was added to the wells D1 to D12 in sequence, and three parallel groups were repeated for each well;
   the fifth group: 50 μL of the 8 μg/mL antibiotic was added to each of the wells E1 to E12, 50 μL of each of the CST with concentration gradients shown in the first group was added to the wells E1 to E12 in sequence, and three parallel groups were repeated for each well;
   the sixth group: 50 μL of the 4 μg/mL antibiotic was added to each of the wells F1 to F12, 50 μL of each of the CST with concentration gradients shown in the first group was added to the wells F1 to F12 in sequence, and three parallel groups were repeated for each well;
   the seventh group: 50 μL of the 2 μg/mL antibiotic was added to each of the wells G1 to G12, 50 μL of each of the CST with concentration gradients shown in the first group was added to the wells G1 to G12 in sequence, and three parallel groups were repeated for each well;
   the eighth group: 50 μL of the 1 μg/mL antibiotic was added to each of the wells H1 to H12, 50 μL of each of the CST with concentration gradients shown in the first group was added to the wells H1 to H12 in sequence, and three parallel groups were repeated for each well;
   the ninth group: 50 μL of the 0.5 μg/mL antibiotic was added to each of the wells I1 to I12, 50 μL of each of the CST with concentration gradients shown in the first group was added to the wells I1 to I12 in sequence, and three parallel groups were repeated for each well;
   the tenth group: 50 μL of the 0.25 μg/mL antibiotic was added to each of the wells J1 to J12, 50 μL of each of the CST with concentration gradients shown in the first group was added to the wells J1 to J12 in sequence, and three parallel groups were repeated for each well;
   the eleventh group: 50 μL of the 0.125 μg/mL antibiotic was added to each of the wells K1 to K12, 50 μL of each of the CST with concentration gradients shown in the first group was added to the wells K1 to K12 in sequence, and three parallel groups were repeated for each well;
   the twelfth group: 50 μL of the 0.0625 μg/mL antibiotic was added to each of the wells L1 to L12, 50 μL of each of the CST with concentration gradients shown in the first group was added to the wells L1 to L12 in sequence, and three parallel groups were repeated for each well.
(6) 1 mL of liquid medium was added to a clear plastic test tube, and the clear plastic test tube was placed on a turbidimeter for zero adjustment. Subsequently, the liquid medium containing the above freshly cultured to-be-measured strain was aspirated and blown uniformly, the turbidity was adjusted to 0.5 MCF turbidity, and the liquid medium containing the above freshly cultured to-be-measured strain was diluted 20 times by using a liquid medium for later use.
(7) 10 μL of the diluted bacterial suspension in each group was taken and sequentially added to the drug-containing liquid medium prepared in step (5) in each well, OD₆₀₀ in each well was detected by using a microplate reader, and the 96-well plate was placed in the incubator at 37° C. and cultured for 16 h. OD₆₀₀ in each well was monitored by using the microplate reader. The lowest drug concentration without bacterial growth (there is no significant change in OD₆₀₀ value during the monitoring) is the MIC of the composition against the bacteria.

The results are shown in Table 2 to Table 8 (where a(b) represents the concentration a corresponding to A and the concentration b corresponding to B in the A-B composition).

TABLE 2

| Composition/ | Candida | | Candida | | Candida | | Candida | | Aspergillus | |
|---|---|---|---|---|---|---|---|---|---|---|
| Single Drug | albicans | | glabrata | | tropicalis | | krusei | | fumigatus | |
| AmB-CST | 0.125 | (0.25) | 0.25 | (8) | 0.25 | (1) | 0.5 | (16) | 0.25 | (1) |
| Nys-CST | 8 | (1) | 1 | (4) | 1 | (0.25) | 0.5 | (8) | 0.5 | (0.25) |
| Mnc-CST | 8 | (1) | 0.25 | (4) | 1 | (1) | 1 | (2) | 0.0625 | (1) |
| Ket-CST | 4 | (0.5) | 0.25 | (0.25) | 1 | (2) | 4 | (1) | 0.125 | (0.25) |
| Flu-CST | 1 | (2) | 4 | (4) | 0.0625 | (1) | 1 | (2) | 1 | (0.25) |
| Itr-CST | 0.125 | (0.25) | 1 | (1) | 0.25 | (0.5) | 2 | (2) | 0.5 | (0.5) |
| Vor-CST | 0.0625 | (1) | 0.25 | (1) | 0.03125 | (1) | 0.125 | (8) | 0.25 | (1) |
| Pos-CST | 0.125 | (1) | 0.25 | (1) | 0.03125 | (2) | 0.0625 | (8) | 0.125 | (0.25) |
| Fly-CST | 0.25 | (0.125) | 0.125 | (1) | 0.0625 | (0.25) | 2 | (8) | 8 | (0.5) |
| Ter-CST | — | | — | | — | | — | | 0.125 | (1) |
| Cas-CST | 0.03125 | (0.25) | 0.03125 | (0.5) | 0.03125 | (1) | 0.125 | (1) | 4 | (1) |
| Mcf-CST | 0.125 | (1) | 0.125 | (1) | 0.125 | (2) | 0.25 | (0.25) | 0.125 | (1) |
| Anf-CST | 0.125 | (2) | 0.03125 | (0.25) | 0.125 | (0.5) | 0.125 | (1) | 0.03125 | (0.5) |
| Gri-CST | 0.5 | (0.5) | 16 | (1) | 4 | (1) | 2 | (2) | 0.5 | (1) |

| | | | | | |
|---|---|---|---|---|---|
| AmB | 0.5 | 0.5 | 1 | 1 | 0.5 |
| Nys | 32 | 4 | 2 | 2 | 2 |
| Mnc | 64 | 2 | 8 | 8 | 0.5 |
| Ket | 16 | 2 | 8 | 32 | 1 |
| Flu | 4 | 32 | 0.5 | 32 | 32 |
| Itr | 0.5 | 4 | 1 | 16 | 2 |
| Vor | 0.25 | 0.5 | 0.0625 | 0.25 | 0.5 |
| Pos | 0.25 | 1 | 0.0625 | 0.125 | 0.5 |
| Fly | 1 | 0.5 | 0.25 | 16 | 32 |
| Ter | — | — | — | — | 0.5 |
| Cas | 0.125 | 0.0625 | 0.0625 | 0.25 | 32 |
| Mcf | 1 | 1 | 0.5 | 1 | 0.5 |
| Anf | 2 | 0.0625 | 2 | 0.5 | 0.0625 |
| Gri | 64 | 64 | 32 | 16 | 64 |
| CST | 4 | 32 | 8 | 32 | 4 |

TABLE 3

| Composition/ | Aspergillus | | Aspergillus | | Aspergillus | | | |
|---|---|---|---|---|---|---|---|---|
| Single Drug | niger | | flavus | | oryzae | | Fusarium | |
| AmB-CST | 1 | (4) | 0.25 | (1) | 0.5 | (8) | 0.25 | (1) |
| Nys-CST | 1 | (4) | 1 | (2) | 2 | (1) | 4 | (1) |
| Mnc-CST | 2 | (8) | 2 | (1) | — | | 4 | (1) |
| Ket-CST | 0.5 | (4) | 0.125 | (1) | 1 | (0.5) | 0.5 | (0.5) |
| Flu-CST | 4 | (1) | 0.5 | (1) | 1 | (1) | 0.25 | (0.25) |
| Itr-CST | 8 | (1) | 0.5 | (2) | 0.25 | (1) | 4 | (1) |
| Vor-CST | 2 | (8) | 0.0625 | (0.5) | 0.125 | (2) | 1 | (0.5) |
| Pos-CST | — | | 0.0625 | (0.25) | 0.03125 | (8) | 0.5 | (0.5) |
| Fly-CST | 1 | (4) | 1 | (0.5) | 8 | (4) | 4 | (1) |
| Ter-CST | 0.0625 | (0.5) | 4 | (0.5) | 1 | (4) | — | |
| Cas-CST | 0.0625 | (0.5) | 0.03215 | (0.0625) | 1 | (2) | 0.5 | (0.25) |
| Mcf-CST | 0.03125 | (1) | 0.03125 | (1) | 0.03125 | (0.125) | 0.25 | (0.125) |
| Anf-CST | 0.03125 | (1) | 0.03215 | (0.125) | 0.0625 | (0.0625) | 0.25 | (0.125) |

| | | | | | | |
|---|---|---|---|---|---|---|
| Gri-CST | 16 | (8) | — | — | 1 | (1) |

| | | | | |
|---|---|---|---|---|
| AmB | 4 | 1 | 1 | 1 |
| Nys | 4 | 16 | 8 | 16 |
| Mnc | 8 | 8 | — | 32 |
| Ket | 2 | 0.5 | 64 | 64 |
| Flu | 16 | 2 | 4 | 4 |
| Itr | 16 | 2 | 0.5 | 64 |
| Vor | 4 | 0.25 | 0.5 | 8 |
| Pos | — | 0.125 | 0.125 | 2 |
| Fly | 4 | 4 | 64 | 32 |
| Ter | 0.125 | 32 | 64 | — |
| Cas | 1 | 0.0625 | 8 | 2 |
| Mcf | 0.125 | 0.125 | 0.0625 | 0.5 |
| Anf | 0.0625 | 0.0625 | 0.125 | 1 |
| Gri | 64 | — | — | 32 |
| CST | 32 | 8 | 32 | 4 |

TABLE 4

| Composition/ | Cryptococcus | | | | | | Histoplasma | |
|---|---|---|---|---|---|---|---|---|
| Single Drug | neoformans | | Candida | | Mucor | | capsulatum | |
| AmB-CST | 0.25 | (0.25) | 0.5 | (2) | 0.25 | (0.125) | 0.03125 | (4) |
| Nys-CST | 0.5 | (0.5) | 1 | (1) | — | | — | |
| Mnc-CST | 0.03125 | (0.125) | 0.5 | (0.5) | 1 | (1) | — | |
| Ket-CST | 1 | (0.5) | 0.25 | (0.5) | 2 | (2) | — | |
| Flu-CST | 1 | (0.5) | 1 | (0.5) | 8 | (1) | 4 | (1) |
| Itr-CST | 0.0625 | (0.5) | 0.0625 | (0.125) | 1 | (0.125) | 0.03125 | (1) |
| Vor-CST | 0.0625 | (0.03125) | 0.5 | (0.25) | 1 | (1) | 0.0625 | (4) |
| Pos-CST | 0.5 | (0.03125) | 0.125 | (1) | 1 | (1) | 0.0625 | (8) |
| Fly-CST | 1 | (0.5) | 1 | (1) | 4 | (1) | 4 | (4) |

| | | | | | | |
|---|---|---|---|---|---|---|
| Ter-CST | 4 | (0.25) | 4 | (1) | — | — |

| | | | | | | | |
|---|---|---|---|---|---|---|---|
| Cas-CST | — | 2 | (0.5) | 4 | (1) | 0.0625 | (0.2.5) |
| Mcf-CST | — | 8 | (1) | 2 | (1) | 0.25 | (1) |
| Anf-CST | — | 0.5 | (0.125) | 0.125 | (0.5) | 0.25 | (1) |

| | | | | | |
|---|---|---|---|---|---|
| Gri-CST | — | — | 0.25 | (1) | — |

| | | | | |
|---|---|---|---|---|
| AmB | 1 | 1 | 1 | 0.125 |
| Nys | 4 | 16 | — | — |
| Mnc | 2 | 2 | 64 | — |
| Ket | 0.125 | 2 | 64 | — |
| Flu | 4 | 8 | 32 | 16 |
| Itr | 0.5 | 0.125 | 32 | 0.0625 |
| Vor | 0.25 | 2 | 8 | 0.25 |
| Pos | 1 | 1 | 2 | 0.125 |
| Fly | 8 | 8 | 32 | 64 |
| Ter | 32 | 16 | — | — |
| Cas | — | 8 | 16 | 0.25 |
| Mcf | — | 16 | 16 | 1 |
| Anf | — | 2 | 0.25 | 1 |
| Gri | — | — | 1 | — |
| CST | 2 | 4 | 8 | 32 |

TABLE 5

| Composition/ | | | | | Cryptococcus | | | |
|---|---|---|---|---|---|---|---|---|
| Single Drug | Scedosporium | | Coccidioides | | gattii | | Paracoccidioides | |
| AmB-CST | 1 | (4) | 0.03125 | (2) | 1 | (0.5) | 0.25 | (1) |
| Nys-CST | — | | — | | — | | — | |
| Mnc-CST | — | | 0.0625 | (0.0625) | — | | — | |
| Ket-CST | — | | — | | — | | 0.03125 | (0.125) |
| Flu-CST | 4 | (4) | 0.25 | (0.0625) | 0.5 | (0.125) | 1 | (0.25) |
| Itr-CST | 0.0625 | (1) | 0.0625 | (0.5) | 0.125 | (0.0625) | 0.03215 | (0.25) |
| Vor-CST | 0.125 | (1) | 0.03125 | (0.25) | 0.0625 | (0.0625) | 0.25 | (0.25) |
| Pos-CST | 1 | (0.25) | 1 | (0.5) | 0.125 | (0.0625) | 0.5 | (0.25) |
| Fly-CST | — | | — | | 0.0625 | (0.0625) | — | |
| Ter-CST | 2 | (4) | 0.25 | (0.25) | 0.5 | (0.25) | 0.0625 | (0.0625) |
| Cas-CST | 4 | (1) | 1 | (0.5) | 1 | (0.5) | 4 | (2) |
| Mcf-CST | 4 | (2) | 2 | (2) | 2 | (0.5) | 1 | (1) |
| Anf-CST | 2 | (2) | 2 | (1) | 1 | (1) | 1 | (1) |

| | | | | |
|---|---|---|---|---|
| Gri-CST | — | — | — | — |
| AmB | 4 | 0.0625 | 2 | 1 |
| Nys | — | — | — | — |
| Mnc | — | 0.125 | — | — |
| Ket | — | — | — | 0.0625 |
| Flu | 16 | 0.5 | 4 | 4 |
| Itr | 0.25 | 0.5 | 0.25 | 0.0625 |
| Vor | 0.5 | 0.125 | 0.25 | 1 |
| Pos | 8 | 2 | 0.25 | 1 |
| Fly | — | — | 0.5 | — |
| Ter | 8 | 1 | 0.5 | 0.5 |
| Cas | 32 | 16 | 16 | 16 |
| Mcf | 32 | 8 | 16 | 8 |
| Anf | 32 | 8 | 16 | 8 |
| Gri | — | — | — | — |
| CST | 32 | 8 | 8 | 16 |

TABLE 6

| Composition/ | Klebsiella | | Acinetobacter | | Pseudomonas | | Enterinvasive | |
|---|---|---|---|---|---|---|---|---|
| Single Drug | pneumoniae | | baumannii | | aeruginosa | | Escherichia coli | |
| Ben-CST | 4 | (1) | — | | — | | 0.0625 | (2) |
| Amo-CST | 4 | (1) | — | | 0.25 | (1) | 0.0625 | (4) |
| Amp-CST | 2 | (0.25) | — | | 1 | (4) | 0.5 | (1) |
| Cfd-CST | 0.25 | (0.25) | 2 | (0.5) | — | | 0.5 | (0.5) |
| Cfa-CST | 0.125 | (0.125) | 4 | (1) | 4 | (0.25) | 0.25 | (2) |
| Cef-CST | 0.0625 | (2) | 2 | (0.5) | 0.0625 | (0.5) | 0.125 | (4) |
| Imi-CST | 0.0625 | (1) | 2 | (0.25) | 0.0625 | (1) | 0.25 | (2) |
| Ami-CST | 0.0625 | (0.25) | 1 | (0.25) | 1 | (1) | 2 | (0.5) |
| Gen-CST | 0.125 | (1) | 8 | (4) | 0.125 | (1) | 8 | (4) |
| Dox-CST | 1 | (4) | 8 | (8) | 0.5 | (0.125) | 0.25 | (4) |
| Tet-CST | 0.03125 | (1) | 0.0625 | (1) | 1 | (2) | 0.0625 | (1) |
| Min-CST | 0.03125 | (0.03215) | 4 | (1) | 2 | (4) | 0.5 | (0.5) |
| Ery-CST | 1 | (0.25) | 0.5 | (0.5) | 32 | (4) | 16 | (16) |
| Cla-CST | 16 | (4) | 1 | (0.25) | 4 | (4) | 4 | (8) |
| Azi-CST | 0.25 | (0.25) | 8 | (4) | 0.5 | (0.50) | 1 | (0.25) |
| Sul-CST | 8 | (4) | 0.5 | (4) | 4 | (0.5) | 4 | (1) |
| Cip-CST | 0.125 | (1) | 0.0625 | (0.125) | 0.0625 | (0.0625) | 0.25 | (8) |
| Lev-CST | 2 | (2) | 0.5 | (0.025) | 0.25 | (2) | 0.25 | (16) |
| Met-CST | 1 | (0.5) | 0.125 | (2) | — | | — | |
| Cli-CST | 0.125 | (0.125) | — | | 0.5 | (0.5) | 0.125 | (1) |
| Fos-CST | 0.125 | (0.125) | 1 | (1) | 1 | (1) | 0.03125 | (0.25) |

| | | | | |
|---|---|---|---|---|
| Ben | 16 | 64 | — | 64 |
| Amo | 64 | — | 2 | 0.125 |
| Amp | 64 | — | 4 | 8 |
| Cfd | 2 | 32 | — | 4 |
| Cfa | 0.5 | 32 | 8 | 4 |
| Cef | 0.25 | 64 | 16 | 2 |
| Imi | 0.25 | 16 | 1 | 2 |
| Ami | 1 | 64 | 4 | 16 |
| Gen | 32 | 64 | 1 | 64 |
| Dox | 2 | 1 | 16 | 0.5 |
| Tet | 0.0625 | 32 | 32 | 2 |
| Min | 1 | 2 | 16 | 4 |
| Ery | 4 | 8 | 64 | 32 |
| Cla | 64 | 32 | 8 | 8 |
| Azi | 2 | 16 | 4 | 16 |
| Sul | 64 | 64 | 32 | 32 |
| Cip | 2 | 32 | 0.25 | 4 |
| Lev | 4 | 2 | 4 | 0.5 |
| Met | 16 | 16 | — | — |
| Cli | 2 | — | 4 | 0.5 |
| Fos | 16 | 64 | 8 | 1 |
| CST | 8 | 8 | 8 | 32 |

TABLE 7

| Composition/ | Enteroaggregative | | | | | | Streptococcus | |
|---|---|---|---|---|---|---|---|---|
| Single Drug | Escherichia coli | | Salmonella | | Shigella | | pneumoniae | |
| Ben-CST | 1 | (4) | 4 | (4) | 2 | (2) | 1 | |
| Amo-CST | — | | 2 | (2) | 1 | (1) | 0.125 | (2) |
| Amp-CST | 0.59 | (0.5) | 0.5 | (2) | 0.5 | (1) | 0.25 | (1) |
| Cfd-CST | 1 | (4) | 0.125 | (1) | 0.0625 | (2) | 0.125 | (1) |
| Cfa-CST | 2 | (4) | 2 | (2) | 0.0625 | (4) | 0.25 | (0.25) |
| Cef-CST | 0.5 | (0.5) | 1 | (0.5) | 0.03125 | (1) | 2 | (2) |
| Imi-CST | 2 | (1) | 0.0625 | (4) | 0.0625 | (1) | 0.125 | (0.125) |
| Ami-CST | 0.5 | (0.25) | 0.5 | (1) | 0.25 | (0.25) | 0.5 | (0.25) |
| Gen-CST | 4 | (4) | 0.03125 | (4) | 0.03125 | (4) | 4 | (2) |
| Dox-CST | 1 | (8) | 0.0625 | (1) | 2 | (1) | 0.25 | (0.125) |
| Tet-CST | 1 | (1) | 2 | (2) | 0.5 | (4) | 1 | (0.5) |
| Min-CST | 0.5 | (0.5) | 2 | (4) | 4 | (4) | 2 | (1) |
| Ery-CST | 0.125 | (1) | 1 | (4) | 0.25 | (2) | 0.25 | (1) |
| Cla-CST | 0.25 | (4) | 0.25 | (0.25) | 16 | (16) | 0.125 | (2) |
| Azi-CST | 16 | (8) | 0.125 | (4) | 1 | (1) | 0.125 | (1) |
| Sul-CST | 2 | (4) | 0.5 | (2) | — | | — | |
| Cip-CST | 0.25 | (8) | 0.125 | (2) | 0.03125 | (1) | 0.0625 | (0.0625) |
| Lev-CST | 0.125 | (2) | 0.0625 | (0.25) | 0.03125 | (16) | 0.0625 | (2) |
| Met-CST | 2 | (0.5) | 16 | (2) | — | | — | |
| Cli-CST | 1 | (1) | 4 | (2) | 32 | (8) | 0.125 | (2) |
| Fos-CST | 0.0625 | (4) | 0.125 | (0.125) | 0.03125 | (4) | 4 | (2) |

| | | | | |
|---|---|---|---|---|
| Ben | 32 | 8 | 16 | 1 |
| Amo | — | 32 | 4 | 64 |
| Amp | 16 | 4 | 2 | 2 |
| Cfd | 8 | 4 | 0.125 | 2 |
| Cfa | 16 | 16 | 0.5 | 8 |
| Cef | 32 | 64 | 0.0625 | 4 |
| Imi | 8 | 0.125 | 0.125 | 32 |
| Ami | 4 | 4 | 2 | 2 |
| Gen | 8 | 0.25 | 0.5 | 16 |
| Dox | 64 | 0.5 | 4 | 0.5 |
| Tet | 64 | 16 | 2 | 4 |
| Min | 16 | 16 | 8 | 4 |
| Ery | 4 | 16 | 64 | 8 |
| Cla | 4 | 1 | 32 | 16 |
| Azi | 32 | 2 | 4 | 2 |
| Sul | 32 | 4 | — | — |
| Cip | 1 | 0.5 | 0.0625 | 1 |
| Lev | 2 | 0.25 | 0.0625 | 2 |
| Met | 8 | 32 | — | — |
| Cli | 4 | 16 | 64 | 1 |
| Fos | 0.25 | 2 | 0.0625 | 16 |
| CST | 16 | 8 | 32 | 4 |

TABLE 8

| Composition/ | Staphylococcus | | Mycobacterium | | Clostridioides | |
|---|---|---|---|---|---|---|
| Single Drug | aureus | | tuberculosis | | difficile | |
| Ben-CST | 0.0625 | (1) | 32 | (16) | — | |
| Amo-CST | 0.25 | (2) | 8 | (16) | 4 | (32) |
| Amp-CST | 0.5 | (0.5) | — | | 1 | |
| Cfd-CST | 0.125 | (0.5) | 8 | (4) | — | |
| Cfa-CST | 4 | (4) | 4 | (8) | — | |
| Cef-CST | 2 | (0.125) | 8 | (8) | 0.5 | (0.5) |
| Imi-CST | 0.0625 | (0.5) | 0.5 | (4) | 4 | (16) |
| Ami-CST | 4 | (8) | 0.125 | (1) | — | |
| Gen-CST | 0.125 | (2) | 0.03125 | (0.5) | 8 | (8) |
| Dox-CST | 16 | (8) | 0.125 | (2) | 0.0125 | (16) |
| Tet-CST | 0.125 | (0.125) | 0.5 | (4) | 1 | (4) |
| Min-CST | 0.03125 | (4) | 0.125 | (2) | 2 | (4) |
| Ery-CST | 8 | (8) | 0.25 | (16) | 2 | (16) |
| Cla-CST | 2 | (8) | — | | 32 | (32) |
| Azi-CST | 16 | (4) | 0.0625 | (2) | 2 | |
| Sul-CST | 0.5 | (4) | 2 | (2) | — | |
| Cip-CST | 0.125 | (1) | 0.5 | (0.125) | 4 | (8) |

| Lev-CST | 4 | (8) | — | 0.0625 | (8) |
|---|---|---|---|---|---|
| Met-CST | — | | — | 0.0625 | (2) |
| Cli-CST | 0.0625 | (0.5) | — | 0.125 | (8) |

| | | | | |
|---|---|---|---|---|
| Fos-CST | 16 | (16) | — | — |

| | | | |
|---|---|---|---|
| Ben | 0.125 | 64 | — |
| Amo | 2 | 32 | 8 |
| Amp | 4 | — | 1 |
| Cfd | 1 | 64 | — |
| Cfa | 8 | 64 | — |
| Cef | 4 | 64 | 8 |
| Imi | 1 | 4 | 8 |
| Ami | 8 | 2 | — |
| Gen | 1 | 0.125 | 32 |
| Dox | 64 | 0.25 | 0.25 |
| Tet | 1 | 4 | 16 |
| Min | 0.125 | 1 | 64 |
| Ery | 64 | 0.5 | 4 |
| Cla | 8 | — | 64 |
| Azi | 64 | 0.25 | 2 |
| Sul | 4 | 8 | — |
| Cip | 0.25 | 2 | 8 |
| Lev | 16 | — | 64 |
| Met | — | — | 0.125 |
| Cli | 0.5 | — | 8 |
| Fos | 32 | — | — |
| CST | 32 | 32 | 64 |

As can be seen from the data results in Table 2 to Table 8, the use of CST in combination with multiple types of antibiotics improves the antimicrobial activity of the antibiotics against various types of fungi and bacteria. Compared with the antibiotic alone or CST alone, the composition significantly reduces the MIC of the antibiotic, effectively broadens the therapeutic window of the drug and improves the safety of the treatment.

### Example 3

### Calculation of FICIs

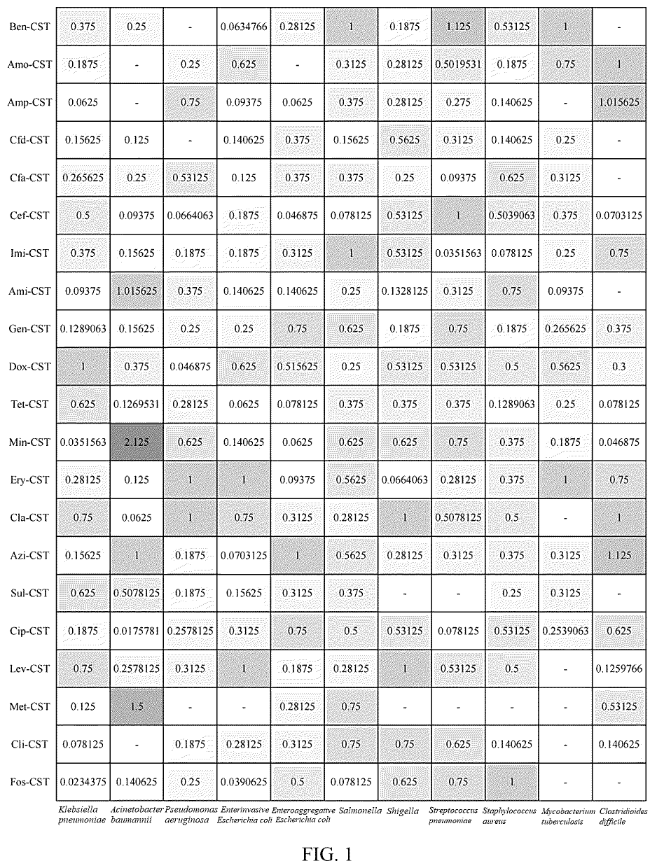

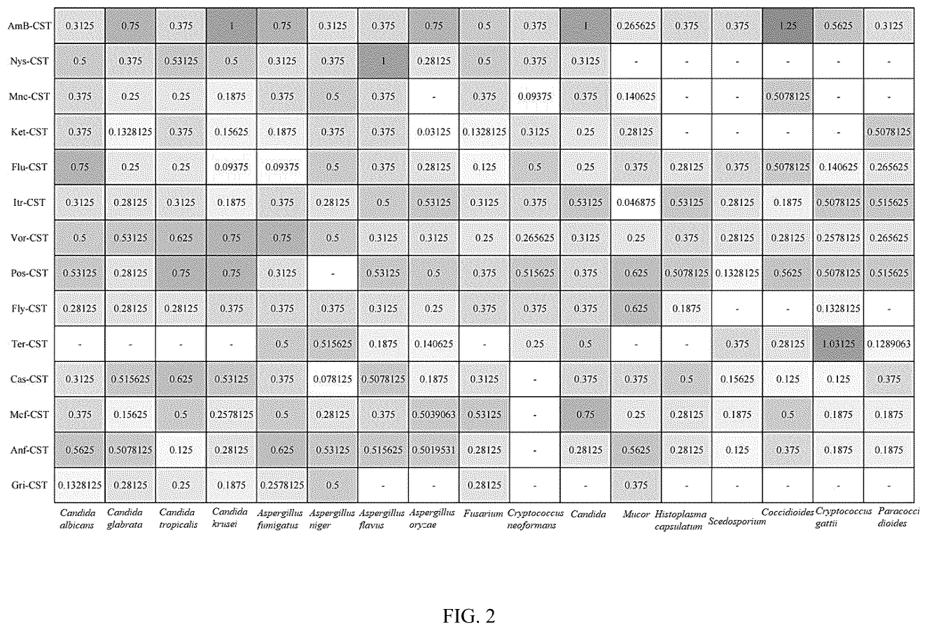

The results were determined according to FICI=MIC (antibiotic in combination)/MIC (antibiotic alone)+MIC (CST in combination)/MIC (CST alone): an effect between CST and the antibiotic was determined as a synergistic effect according to FICI≤0.5, determined as an additive effect according to 0.5<FICI≤1, determined as an irrelevant effect according to 1<FICI≤2 and determined as an antagonistic effect according to FICI>2. The results are shown in FIGS. 1 and 2.

As can be seen from the results in FIGS. 1 and 2, among the combined compositions involved in the present application, CST and most antibiotics exhibit a significant synergistic effect, effectively inhibiting the growth of fungi and bacteria; the Flu-CST combination exhibits an excellent synergistic anti-Aspergillus fumigatus effect, and 0.25 μg/mL CST can reduce the MIC of fluconazole to 1/32 of the original MIC, both of which has an antimicrobial sensitization effect significantly superior to that of an antibiotic adjuvant already found in the related art. In addition, some results indicate that there is an antagonism phenomenon between CST and some antibiotics. Minocycline and CST exhibit an antagonistic effect in inhibiting a growth process of Acinetobacter baumannii. The present application provides a guiding effect for the combined use of CST and different antibiotics to treat different bacterial and fungal infections.

### Example 4

### Molecular Docking Results of CST and Antimicrobial Targets CYP51

Researches on semi-flexible docking of 3D structures (PDB ID: 6CR2) of CYP51 proteins (NCBI Gene ID: 13121) and CST (PubChem CID: 122724) molecules were performed by using AutoDock 4.2.6 software. For the docking box, the size was set to 100×100×100 points, the spacing was set to 0.375 Å, and the active site of the enzyme was used as the center. The result with the lowest binding energy and the largest number of hydrogen bonds was selected as the final result. Images were processed by using Discovery Studio 2021 (Biovia, San Diego, CA, USA) software, and binding modes such as intermolecular hydrogen bonds between micromolecules and proteins were displayed in two-dimensional and three-dimensional forms.

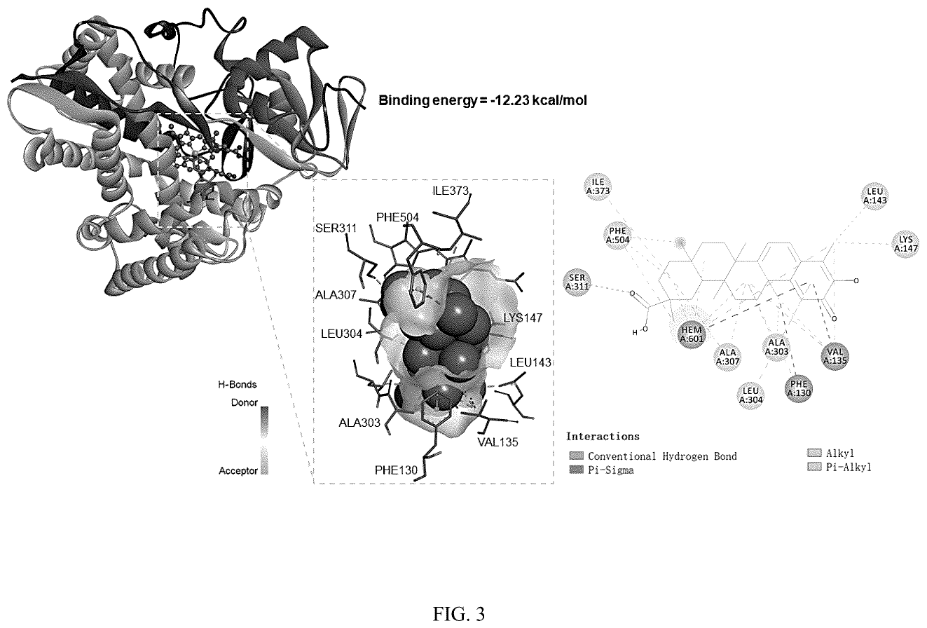

The docking results are shown in FIG. 3. CST can fit well into active pockets of the CYP51 proteins, and the binding energy was −12.23 kcal/mol. A relatively strong intermolecular hydrogen bond can be formed by CST and a SER311 amino acid residue, and there are π-σ and π-alkyl interactions between CST and iron porphyrin, effectively promoting the binding of CST to the CYP51 proteins. As the most widely distributed member of cytochrome P450 family, CYP51 is a key enzyme in a synthesis process of biological sterols. CST can specifically bind to the CYP51 proteins and inhibit the synthesis of sterols in fungi, exerting antifungal activity.

### Example 5

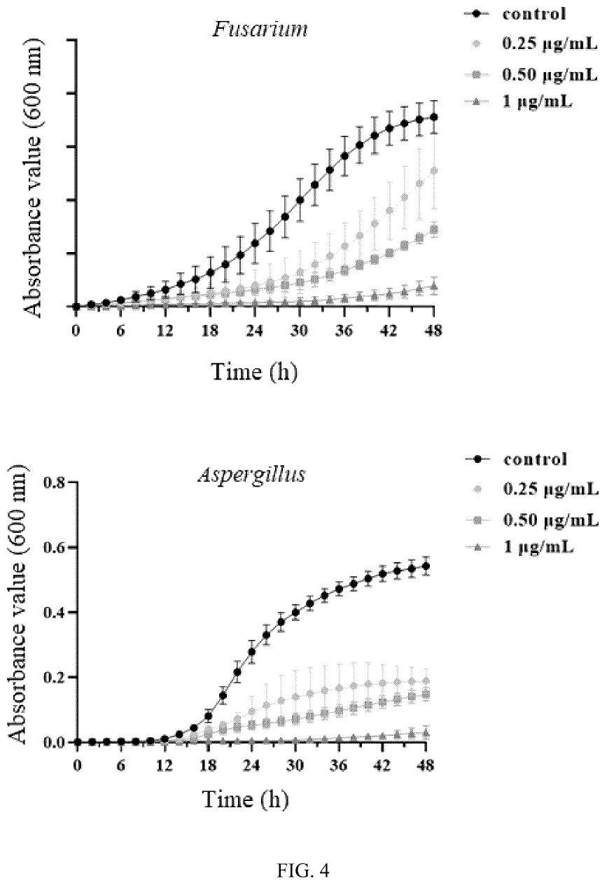

This example provides a combined composition prepared through physical mixing of fluconazole with CST at a molar ratio of 2.72:1. The inhibitory effects of the combined composition against Fusarium and Aspergillus fumigatus were evaluated.
(1) Aspergillus fumigatus and Fusarium fungal solutions cultured overnight were diluted to a spore concentration of 1×10⁶ CFU/mL by using RPMI 1640 media for later use.
(2) 100 μL of the diluted fungal solution was added to each well of a sterile 96-well plate, and then 100 μL of the combined composition solution was added to each well so that final total concentrations of fluconazole were 0.25 μg/mL, 0.5 μg/mL and 1 μg/mL, respectively. PBS was set as a blank control group in each group.
(3) Subsequently, the sterile 96-well plate was placed in an incubator and subjected to static culture at 37° C. An A value in each well was measured every 2 h at a wavelength of 600 nm by using a multifunctional microplate reader and continuously recorded for 48 h. The results are shown in FIG. 4.

As can be seen from FIG. 4, the combined composition exhibits a concentration-dependent antifungal effect. With a concentration of 1 μg/mL, the combined composition can effectively inhibit the growth of Fusarium and Aspergillus fumigatus.

### Example 6

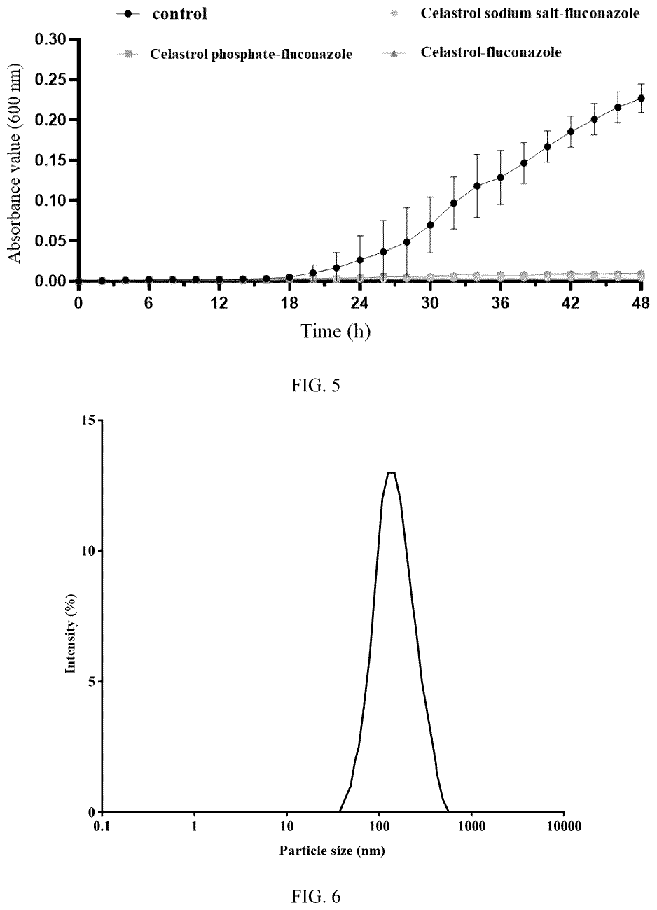

This example provides multiple types of combined compositions prepared through physical mixing of fluconazole with CST or a pharmaceutical salt thereof (celastrol sodium salt or celastrol phosphate) at a molar ratio of 2.72:1. The inhibitory effects of the combined compositions against Aspergillus fumigatus were evaluated.
(1) An Aspergillus fumigatus fungal solution cultured overnight was diluted to a spore concentration of 1×10⁶ CFU/mL by using an RPMI 1640 medium for later use.
(2) 100 μL of the diluted fungal solution was added to each well of a sterile 96-well plate, and 100 μL of the combined composition solution in each group was added so that a final total concentration of fluconazole was 1 μg/mL after the preparation. PBS was set as a blank control group.
(3) Subsequently, the sterile 96-well plate was placed in an incubator and subjected to static culture at 37° C. An A value in each well was measured every 2 h at a wavelength of 600 nm by using a multifunctional microplate reader and continuously recorded for 48 h. The results are shown in FIG. 5.

As can be seen from FIG. 5, the pharmaceutical salt of CST and CST have the same antifungal effect. With a concentration of 1 μg/mL, both the pharmaceutical salt of CST and CST can effectively inhibit the growth of Aspergillus fumigatus.

### Preparation Example 1

This preparation example provides carrier-free nanoparticles, and a method for preparing the carrier-free nanoparticles is described below.
(1) 2 mg of CST and 3 mg of fluconazole were dissolved in 1 mL of dimethylsulfoxide and mixed thoroughly.
(2) Under a condition of stirring, the above mixed liquid was added dropwise to 10 mL of deionized water.
(3) The obtained mixed liquid was placed in a magnetic stirrer and stirred for 10 min to obtain initial carrier-free nanoparticles.
(4) Ultrafiltration centrifugation was performed at 8000 g/15 min to obtain the carrier-free nanoparticles.

### Preparation Example 2

This preparation example provides carrier-free nanoparticles, and a method for preparing the carrier-free nanoparticles is described below.
(1) 2 mg of CST and 2 mg of fluconazole were dissolved in 1 mL of methanol and mixed thoroughly.
(2) Under a condition of stirring, the above mixed liquid was added dropwise to 5 mL of deionized water.
(3) The obtained mixed liquid was placed in a magnetic stirrer and stirred for 15 min to obtain initial carrier-free nanoparticles.
(4) Ultrafiltration centrifugation was performed at 10000 g/10 min to obtain the carrier-free nanoparticles.

### Preparation Example 3

This preparation example provides carrier-free nanoparticles, and a method for preparing the carrier-free nanoparticles is described below.
(1) 2 mg of CST and 3 mg of fluconazole were dissolved in 1.5 mL of methanol and mixed thoroughly.
(2) Under a condition of stirring, the above mixed liquid was added dropwise to 6 mL of deionized water.
(3) The obtained mixed liquid was placed in a magnetic stirrer and stirred for 25 min to obtain initial carrier-free nanoparticles.
(4) Ultrafiltration centrifugation was performed at 8000 g/15 min to obtain the carrier-free nanoparticles.

### Preparation Example 4

This preparation example provides carrier-free nanoparticles, and a method for preparing the carrier-free nanoparticles is described below.
(1) 2 mg of CST and 5 mg of amphotericin B were dissolved in 2 mL of dimethylsulfoxide and mixed thoroughly.
(2) Under a condition of stirring, the above mixed liquid was added dropwise to 6 mL of deionized water.
(3) The obtained mixed liquid was placed in a magnetic stirrer and stirred for 25 min to obtain initial carrier-free nanoparticles.
(4) Ultrafiltration centrifugation was performed at 8000 g/15 min to obtain the carrier-free nanoparticles.

### Preparation Example 5

This preparation example provides a composite liposome, and a method for preparing the composite liposome is described below.
(1) 1 mg of CST, 2 mg of amphotericin B, 3.5 mg of 1,2-dioleoyl-SN-glycero-3-phosphoethanolamine, 1.0 mg of DSPE-mPEG2000 and 0.28 mg of cholesterol were co-dissolved in 2 mL of methanol, placed in a 10 mL of round-bottom flask, mixed thoroughly and uniformly, and subjected to rotary evaporation under a condition of 30° C. water bath to form a film.
(2) 4 mL of deionized water was added, and hydration was performed for 15 min to obtain an initial composite liposome.
(3) The initial composite liposome was subjected to probe ultrasonication (200 w, 5 min, ultrasound was turned on for 3 s and turned off for 3 s) and passed through 400 nm and 200 nm filter membranes in sequence for gradient extrusion, and finally, the composite liposome was obtained.

### Preparation Example 6

This preparation example provides a composite liposome, and a method for preparing the composite liposome is described below.
(1) 1.5 mg of CST, 1 mg of fluconazole, 4.5 mg of 1,2-dioleoyl-SN-glycero-3-phosphoethanolamine, 1.5 mg of DSPE-mPEG2000 and 0.35 mg of cholesterol were co-dissolved in 2 mL of methanol, placed in a 10 mL of round-bottom flask, mixed thoroughly and uniformly, and subjected to rotary evaporation under a condition of 30° C. water bath to form a film.
(2) 4 mL of deionized water was added, and hydration was performed for 25 min to obtain an initial composite liposome.
(3) The initial composite liposome was subjected to probe ultrasonication (200 w, 5 min, ultrasound was turned on for 3 s and turned off for 3 s) and passed through 400 nm and 200 nm filter membranes in sequence for gradient extrusion, and finally, the composite liposome was obtained.

### Preparation Example 7

This preparation example provides a composite liposome, and a method for preparing the composite liposome is described below.
(1) 2 mg of CST, 2 mg of amphotericin B, 3.5 mg of DOPE, 1.0 mg of DSPE-mPEG2000 and 0.28 mg of cholesterol were co-dissolved in 2 mL of methanol, placed in a 10 mL of round-bottom flask, mixed thoroughly and uniformly, and subjected to rotary evaporation under a condition of 30° C. water bath to form a film.
(2) 4 mL of deionized water was added, and hydration was performed for 15 min to obtain an initial composite liposome.
(3) The initial composite liposome was subjected to probe ultrasonication (200 w, 5 min, ultrasound was turned on for 3 s and turned off for 3 s) and passed through 400 nm and 200 nm filter membranes in sequence for gradient extrusion, and finally, the composite liposome was obtained.

### Preparation Example 8

This preparation example provides a composite liposome, and a method for preparing the composite liposome is described below.
(1) 2 mg of CST and 2 mg of amphotericin B were dissolved in 1 mL of methanol and mixed thoroughly.
(2) Under a condition of stirring, the above mixed liquid was added dropwise to 5 mL of deionized water.
(3) The obtained mixed liquid was placed in a magnetic stirrer and stirred for 15 min to obtain initial carrier-free nanoparticles.
(4) 15 mg of DPPC and 2 mg of cholesterol were dissolved in 3 mL of CHC₃ and mixed thoroughly, and CHCl₃ was removed through rotary evaporation (45° C., 15 min). Subsequently, the carrier-free nanoparticles solution obtained in step (3) were added and hydrated thoroughly (25° C., 30 min).
(5) Subsequently, the composite liposome was obtained through probe ultrasonication (15 min, 100 w) and gradient extrusion.

### Preparation Example 9

This preparation example provides a composite liposome, and a method for preparing the composite liposome is described below.
(1) 1 mg of CST and 4 mg of fluconazole were dissolved in 0.5 mL of methanol and mixed thoroughly.
(2) Under a condition of stirring, the above mixed liquid was added dropwise to 2.5 mL of deionized water.
(3) The obtained mixed liquid was placed in a magnetic stirrer and stirred for 12 min to obtain initial carrier-free nanoparticles.
(4) Synthesis of fluorinated DSPE-PEG2000

4,4,4-Trifluorobutanoic acid (35.5 mg), 1-ethyl-3-(3-dimethylaminopropyl) carbodiimide hydrochloride (EDC:HCl, 71.9 mg), N-hydroxysuccinimide (NHS, 28.8 mg) and 4-dimethylaminopyridine (DMAP, 3.05 mg) were weighed and co-dissolved in 10 mL of CHCl₃ (1 mL), and the mixture was placed on ice and stirred to react for 4 h to obtain a system 1. Subsequently, 139.5 mg of DSPE-PEG-NH2 was dissolved in CHCl₃ (1 mL), the system 1 was added, and the mixture was stirred for 20 h at room temperature to obtain a system 2. After the organic solvent in the system 2 was removed through vacuum drying, 5 mL of ultrapure water was added to obtain a system 3, and the system 3 was placed in a dialysis bag and dialysed for 2 days. After 2 days, the product in the dialysis bag was lyophilized to obtain fluorinated DSPE-PEG2000 as a powder.
(5) 10 mg of DPPC, 5 mg of DOPE, 1 mg of fluorinated DSPE-PEG2000 and 1 mg of cholesterol were dissolved in 3 mL of CHCl₃ and mixed thoroughly, and CHCl₃ was removed through rotary evaporation (45° C., 20 min). Subsequently, the carrier-free nanoparticle solution obtained in step 3 was added and hydrated thoroughly (25° C., 15 min). Subsequently, the composite liposome was obtained through probe ultrasonication (15 min, 150 w) and gradient extrusion.

### Preparation Example 10

This preparation example provides composite polymer nanoparticles, and a method for preparing the composite polymer nanoparticles is described below.
(1) 10 mg PLGA was dissolved in 1 mL of dichloromethane.
(2) 0.5 mg of CST and 5 mg of amphotericin B were dissolved in dichloromethane, and the mixture was added dropwise to 3 mL of deionized water containing 5% polysorbate 80.
(3) The PLGA solution in step (1) was slowly dripped into the aqueous solution in step (2), and the mixture was subjected to ultrasonication for 2 min by using a 150 w ultrasonic disruptor to obtain an initial emulsion.
(4) The initial emulsion was slowly added to 10 mL of deionized water containing 50% polysorbate 80, and the mixture was subjected to ultrasonication for 3 min by using the 150 w ultrasonic disruptor and mixed thoroughly for 12 h to obtain a double emulsion.
(5) Dichloromethane in the double emulsion was removed by using a rotary evaporator to obtain the composite polymer nanoparticles (AmB-CST NPs).

### Test Example 1

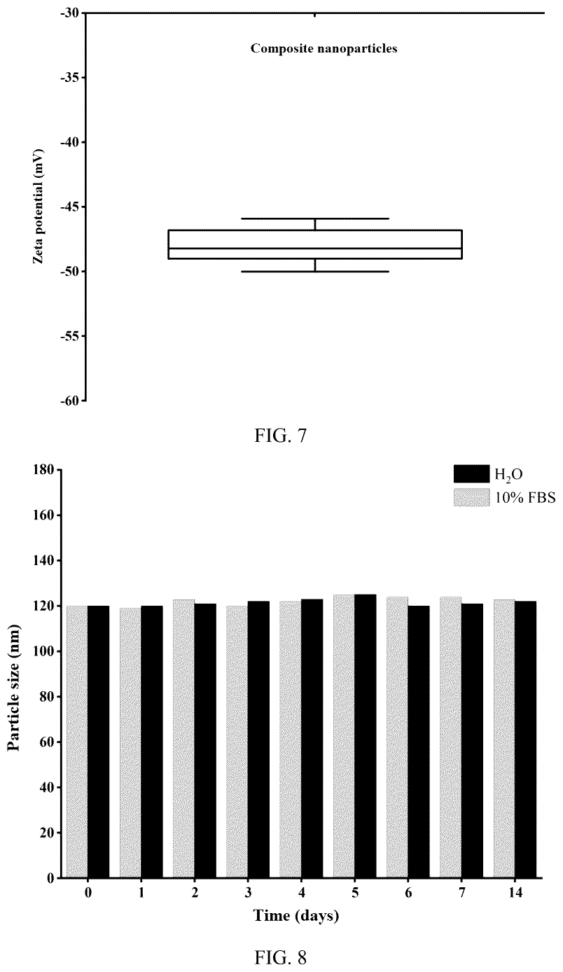

Hydrated particle sizes and potentials of the carrier-free nanoparticles prepared in Preparation Example 1 were measured by using a dynamic light scattering instrument (Zetasizer NanoZS). The results are shown in FIGS. 6 and 7. The carrier-free nanoparticle has a hydrated particle size of about 120 nm, a polydispersity index of 0.15 and a surface charge of −47.5 mV.

### Test Example 2

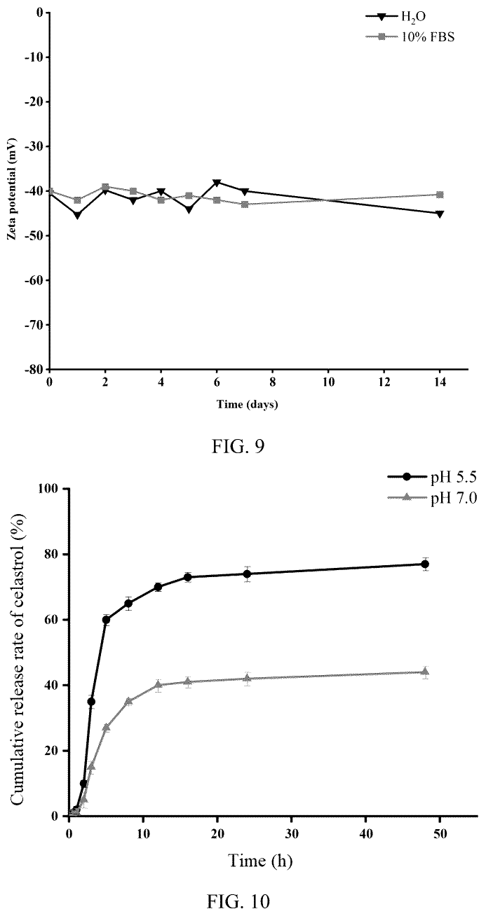

The carrier-free nanoparticles prepared in Preparation Example 2 were added to a PBS solution containing 10% FBS or ultrapure water, and hydrated particle sizes and potentials were measured on days 0, 1, 2, 3, 4, 5, 6, 7 and 14. As shown in FIGS. 8 and 9, it indicates that the carrier-free nanoparticles have good physical stability.

### Test Example 3

The encapsulation efficiency and drug-loading efficiency of the carrier-free nanoparticles prepared in Preparation Example 2 were calculated. Full-wavelength scanning was performed on absorption peaks of CST, fluconazole and the carrier-free nanoparticles by using an ultraviolet spectrophotometer. The maximum absorption peaks of CST, fluconazole and the carrier-free nanoparticles are 423 nm, 251 nm and 425 nm, respectively. A concentration of fluconazole was calibrated through liquid chromatography, and a concentration of CST was calibrated by using an ultraviolet-visible absorption spectrum. Subsequently, contents of CST and fluconazole in the carrier-free nanoparticles were measured to calculate the encapsulation efficiency and the drug-loading efficiency. It can be seen that CST has an encapsulation efficiency of 87.8% and a drug-loading efficiency of 72.7%, and fluconazole has an encapsulation efficiency of 83.6% and a drug-loading efficiency of 27.3%.

### Test Example 4

An in vitro release ability of the carrier-free nanoparticles prepared in Preparation Example 2 in a slightly acidic environment was evaluated. 1 mL of the above carrier-free nanoparticles was taken and added to each dialysis bag with a molecular weight of 3500, and the dialysis bags were placed in 50 mL of a 0.1 M PBS solution (containing 0.5% polysorbate 80) with a pH of 5.5 and 50 mL of a 0.1 M PBS solution (containing 0.5% polysorbate 80) with a pH of 7.0, respectively. 1.5 mL of the PBS solution was taken at different time points, and absorbance values at 423 nm were detected by using an ultraviolet-visible absorption spectrum to measure a concentration of CST. The results are shown in FIG. 10. It can be seen that the carrier-free nanoparticles can be quickly released in the acidic buffer.

### Test Example 5

Antifungal effects of the products prepared in Preparation Examples 3, 4 and 10 were evaluated.

MICs of the carrier-free nanoparticles (recorded as Flu-CST NPs) prepared in Preparation Example 3, the carrier-free nanoparticles (recorded as AmB-CST NPs) prepared in Preparation Example 4, the composite polymer nanoparticles (recorded as AmB-CST NPs) prepared in Preparation Example 10 and free CST, fluconazole (Flu) and amphotericin B (AmB) against Aspergillus fumigatus were tested through the method recorded in Example 1. The use concentration of Flu-CST NPs was based on the concentration of Flu, and the use concentration of AmB-CST NPs was based on the concentration of AmB. The results are shown in Table 9.

TABLE 9

| | Minimum Inhibitory |
|---|---|
| Group | Concentration (μg/mL) |
| Flu-CST NPs | 0.5 |
| AmB-CST NPs (Preparation Example 4) | 0.25 |
| AmB-CST NPs (Preparation Example 10) | 0.25 |
| CST | 4 |
| Flu | 32 |
| AmB | 0.5 |

As can be seen from the results in Table 9, compared with the antibiotic alone or CST alone, the carrier-free nanoparticles or the composite polymer nanoparticles prepared from the antibiotic and CST that are used as raw materials can significantly reduce the MIC of the antibiotic, effectively broadens the therapeutic window of the drug and improves the safety of the treatment.

In addition, effects of the composite liposome (recorded as Flu-CST NPs) prepared in Preparation Example 9, CST and fluconazole (Flu) on cell membranes of Aspergillus fumigatus and drug-resistant strains thereof were verified through a crystal violet biofilm staining experiment.
(1) A SDA medium was prepared, 50 mL of an aqueous solution of chloramphenicol (2 mg/mL) was added, the pH was adjusted to 7.0, the volume of the solution was precisely adjusted to 1000 mL, and the solution was stored at 4° C. after autoclave. To-be-tested strains, including non-drug-resistant Aspergillus fumigatus and clinical drug-resistant strains (Aspergillus fumigatus clinical), were activated on SDA medium plates to induce the formation of conidia and sporangiospores.
(2) A fungal suspension was prepared with a 0.85% NaCl solution (containing 0.01 mL of polysorbate 20). After the fungal suspension was left to stand for 10 min, an upper uniform liquid containing sporangiospores or conidia and mycelial fragments was taken and adjusted to a liquid with a concentration of 1×10⁴ CFU/mL with an RPMI 1640 culture solution.
(3) Then, 1 mL of the fungal suspension was inoculated into each well of a 24-well plate, and PBS, Flu (the final concentration was 0.25 μg/mL), CST (the final concentration was 0.75 μg/mL) and Flu-CST NPs (based on the concentration of Flu, the final concentration was 0.25 μg/mL) were added to the wells, respectively, and the mixtures were incubated for 24 h at 37° C.
(4) The medium was aspirated, and the wells were washed three times with PBS. A biofilm adhered to each well was immobilized with 200 μL of methanol for 15 min at 25° C. and dried for 30 min at 25° C. Each well was further stained with 1 mL of a 0.1% crystal violet solution for 20 min. The well was thoroughly washed to remove the unbound crystal violet solution. Subsequently, 1 mL of a 95% ethanol solution was added to each well. An absorbance value at A₆₀₀ nm was measured by using a microtiter microplate reader to measure a content of the biofilm.

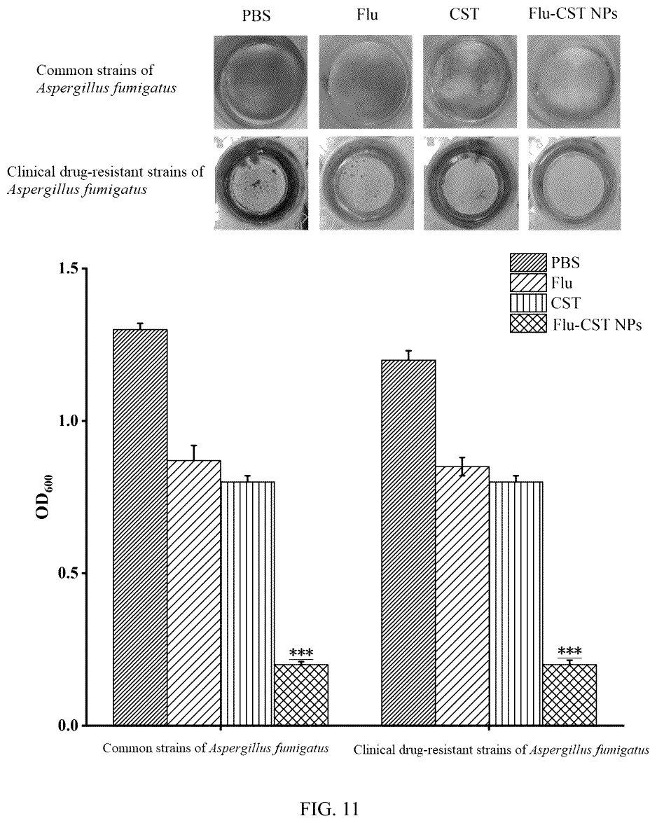

The results are shown in FIG. 11. Compared with Flu and CST, Flu-CST NPs have a more significant effect of inhibiting the formation of the fungal biofilm.

### Test Example 6

The biosafety of the carrier-free nanoparticles prepared in Preparation Examples 3 and 4 were evaluated.

PBS was added to fresh blood extracted from female Balb/c nude mice, and the mixture was mixed gently and uniformly, centrifuged at 3000 rpm for 10 min and washed until the supernatant was colorless. Lower precipitates were diluted with a PBS solution to obtain a red blood cell (RBC) suspension. The carrier-free nanoparticles prepared in Preparation Example 3 (recorded as Flu-CST NPs), the carrier-free nanoparticles prepared in Preparation Example 4 (recorded as AmB-CST NPs), CST, polysorbate 80 and the RBC suspension were incubated for 4 h at 37° C. and centrifuged at 3000 rpm for 10 min. The absorbance of the supernatant at 541 nm was measured by using a microplate reader to calculate a hemolysis rate. The results are shown in FIG. 12. It can be seen that the carrier-free nanoparticles involved in the present application have good blood compatibility.

### Test Example 7

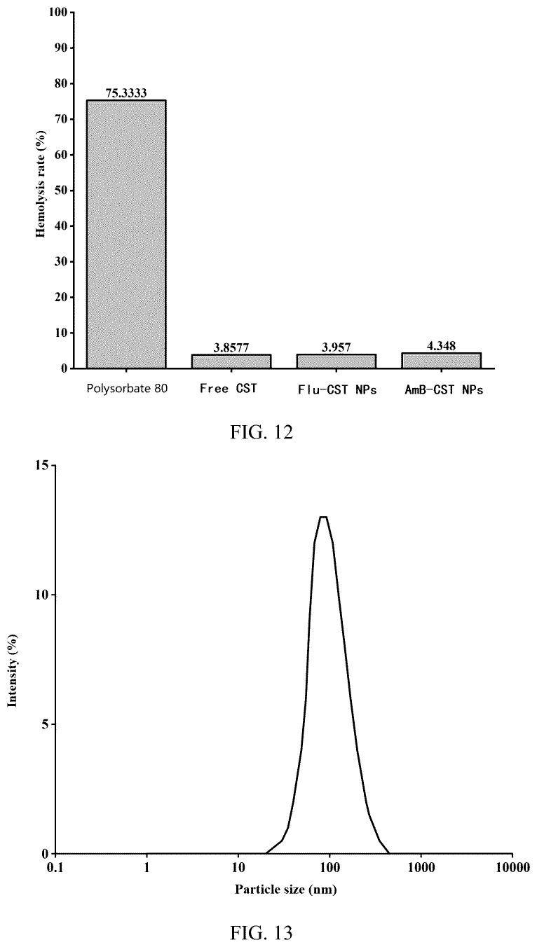

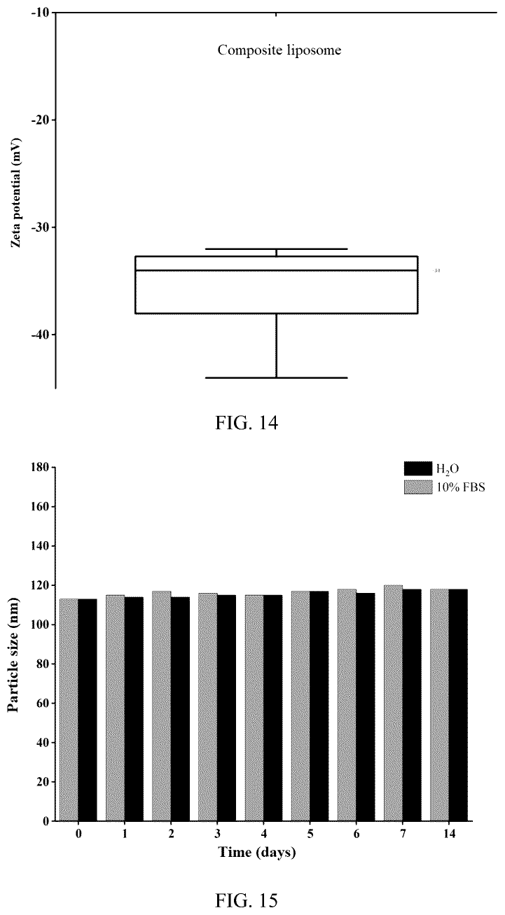

A hydrated particle size and potential of the composite liposome prepared in Preparation Example 5 were measured by using a dynamic light scattering instrument (Zetasizer NanoZS). The results are shown in FIGS. 13 and 14. The composite liposome has a hydrated particle size of about 113 nm, a polydispersity index of 0.18 and a surface charge of −32.7 mV.

### Test Example 8

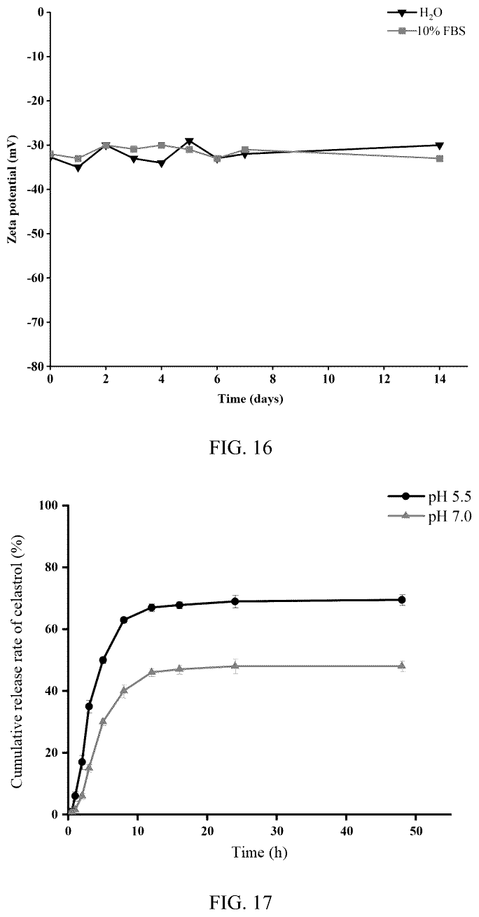

The composite liposome prepared in Preparation Example 7 was added to a PBS solution containing 10% FBS or ultrapure water, and a hydrated particle size and a potential were measured on days 0, 1, 2, 3, 4, 5, 6, 7 and 14. As shown in FIGS. 15 and 16, it indicates that the composite liposome has good physical stability.

### Test Example 9

The encapsulation efficiency and drug-loading efficiency of the composite liposome prepared in Preparation Example 7 were calculated. Full-wavelength scanning was performed on absorption peaks of CST, amphotericin B and the composite liposome by using an ultraviolet spectrophotometer. The maximum absorption peaks of CST, amphotericin B and the composite liposome are 423 nm, 405 nm and 425 nm, respectively. A concentration of amphotericin B was calibrated through liquid chromatography, and a concentration of CST was calibrated by using an ultraviolet-visible absorption spectrum. Subsequently, contents of CST and amphotericin B in the composite liposome were measured to calculate the encapsulation efficiency and the drug-loading efficiency. It can be seen that CST has an encapsulation efficiency of 80.8% and a drug-loading efficiency of 52.3%, and amphotericin B has an encapsulation efficiency of 43.4% and a drug-loading efficiency of 17.9%.

### Test Example 10

An in vitro release ability of the composite liposome prepared in Preparation Example 7 in a slightly acidic environment was evaluated. 1 mL of the above composite liposome was taken and added to each dialysis bag with a molecular weight of 3500, and the dialysis bags were placed in 50 mL of a 0.1 M PBS solution (containing 0.5% polysorbate 80) with a pH of 5.5 and 50 mL of a 0.1 M PBS solution (containing 0.5% polysorbate 80) with a pH of 7.0, respectively. 1.5 mL of the PBS solution was taken at different time points, and absorbance values at 423 nm were detected by using an ultraviolet-visible absorption spectrum to measure a concentration of CST. The results are shown in FIG. 17. It can be seen that the composite liposome can be quickly released in the acidic buffer.

### Test Example 11

Antifungal effects of the products prepared in Preparation Examples 6, 7 and 10 were evaluated.

MICs of the composite liposome (recorded as Flu-CST Lips) prepared in Preparation Example 6, the composite liposome (recorded as AmB-CST Lips) prepared in Preparation Example 7, the composite polymer nanoparticles (recorded as AmB-CST NPs) prepared in Preparation Example 10 and free CST, fluconazole (Flu) and amphotericin B (AmB) against Aspergillus fumigatus were tested through the method recorded in Example 1. The use concentration of Flu-CST Lips was based on the concentration of Flu, and the use concentrations of AmB-CST Lips and AmB-CST NPs were based on the concentration of AmB. The results are shown in Table 10.

TABLE 10

| | Minimum Inhibitory Concentration |
|---|---|
| Group | (μg/mL) |
| Flu-CST Lips | 0.25 |
| AmB-CST Lips | 0.125 |
| AmB-CST NPs | 0.5 |
| CST | 4 |
| Flu | 32 |
| AmB | 0.5 |

As can be seen from the results in Table 10, compared with the antibiotic alone or CST alone, the composite liposome or the composite polymer nanoparticles prepared from the antibiotic and CST that are used as raw materials can significantly reduce the MIC of the antibiotic, and the composite liposome has a more excellent effect. This effectively broadens the therapeutic window of the drug and improves the safety of the treatment.

### Test Example 12

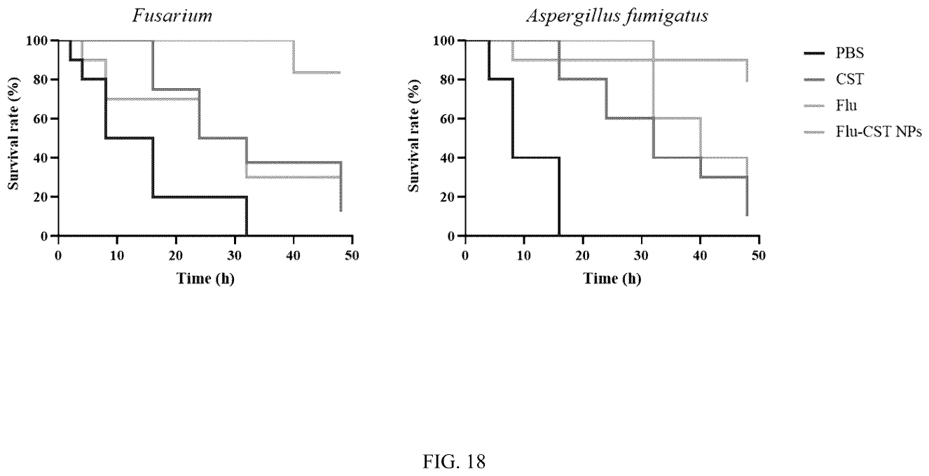

10 μL of 2×10⁸ CFU Fusarium and 10 μL of 2×10⁸ CFU Aspergillus fumigatus were injected to infect Galleria mellonella larvae, respectively, and 10 μL of PBS, 10 mg/mL CST, 30 mg/mL Flu and Flu-CST NPs (the product in Preparation Example 9, and the actual concentration of Flu was 30 mg/mL) were injected into the Galleria mellonella larvae, respectively. Subsequently, the Galleria mellonella larvae were placed in an incubator at 37° C., and survival states of the Galleria mellonella larvae were recorded every 8 h and continuously recorded for 48 h. As shown in FIG. 18, for the Galleria mellonella larvae inoculated with Fusarium and Aspergillus fumigatus, the survival rate of the experimental group treated with CST or Flu alone within 48 h is less than 40%, indicating that using CST or Flu alone cannot effectively treat the fungal infection. After the combined medication treatment, the survival rate of the Galleria mellonella larvae infected with Fusarium reaches 90%, and the survival rate of the Galleria mellonella larvae infected with Aspergillus fumigatus reaches 80%, indicating that the combined composition nanoparticles involved in the present application can effectively treat the fungal infection and significantly improve the survival rate of the Galleria mellonella larvae.

The applicant has stated that although the technical solutions of the present application are described through the examples described above, the present application is not limited to the examples described above, which means that the implementation of the present application does not necessarily depend on the examples described above. It should be apparent to those skilled in the art that any improvements made to the present application, equivalent replacements of raw materials of the product of the present application, additions of adjuvant ingredients, selections of specific manners, etc., all fall within the protection scope and the disclosure scope of the present application.

Although preferred embodiments of the present application are described above in detail, the present application is not limited to details of the preceding embodiments, and various simple modifications can be made to the technical solutions of the present application without departing from the technical concept of the present application. These simple modifications are all within the protection scope of the present application.

Additionally, it is to be noted that if not in collision, specific technical features described in the preceding embodiments can be combined in any suitable manner. To avoid unnecessary repetition, various possible combination manners are no longer described in the present application.

1. A method for treating or preventing fungal and bacterial infections, the method comprising:
administering an effective amount of a drug to subject in need thereof, wherein the drug comprises a combined composition;
wherein active components of the combined composition are an antibiotic and an antibiotic adjuvant; and
wherein the antibiotic adjuvant is celastrol or a pharmaceutical salt thereof.

2. The method according to claim 1, wherein the antibiotic comprises any one or a combination of at least two of amphotericin B, nystatin, miconazole, ketoconazole, fluconazole, itraconazole, voriconazole, posaconazole, flucytosine, terbinafine, caspofungin, micafungin, anidulafungin, griseofulvin, penicillin, amoxicillin, ampicillin, cefradine, cefalexin, ceftazidime, imipenem, amikacin, gentamicin, doxycycline, tetracycline, minocycline, erythromycin, clarithromycin, azithromycin, sulfadiazine, ciprofloxacin, levofloxacin, metronidazole, clindamycin or fosfomycin.

3. The method according to claim 1, wherein the fungi comprise any one or a combination of at least two of Candida albicans, Candida glabrata, Candida tropicalis, Candida krusei, Aspergillus fumigatus, Aspergillus niger, Aspergillus flavus, Aspergillus oryzae, Fusarium, Cryptococcus neoformans, Candida, Mucor, Histoplasma capsulatum, Scedosporium, Coccidioides, Cryptococcus gattii or Paracoccidioides.

4. The method according to claim 1, wherein the bacteria comprise any one or a combination of at least two of Klebsiella pneumoniae, Acinetobacter baumannii, Pseudomonas aeruginosa, Escherichia coli, Salmonella, Shigella, Streptococcus pneumoniae, Staphylococcus aureus, Mycobacterium tuberculosis or Clostridioides difficile.

5. The method according to claim 1, wherein a molar ratio of the antibiotic to the antibiotic adjuvant is 1:(0.001-500).

6. The method according to claim 1, wherein the combined composition is a single compound preparation, a combination of an antibiotic adjuvant and an antibiotic each in separate preparations, or a nanoparticle;
when the combined composition is the combination of the antibiotic adjuvant and the antibiotic each in separate preparations, a mode of administration of the combination of the antibiotic adjuvant and the antibiotic each in separate preparations may be simultaneous administration, cross administration or sequential administration;
wherein when the combined composition is the nanoparticle, the combined composition comprises the following three forms:
in form 1, the combined composition comprises carrier-free nanoparticles formed by an antibiotic and an antibiotic adjuvant;
in form 2, the combined composition comprises a composite liposome prepared from an antibiotic, an antibiotic adjuvant, a lipid and cholesterol that are used as raw materials;
wherein the lipid comprises any one or a combination of at least two of phosphatidylcholine, phosphatidylethanolamine, phosphatidylinositol, phosphatidic acid, cardiolipin, sphingomyelin, phosphatidylserine, DSPE-PEG or fluorine-modified DSPE-PEG;
in form 3, the combined composition comprises composite polymer nanoparticles prepared from an antibiotic, an antibiotic adjuvant and a pharmaceutical degradable polymer that are used as raw materials;
wherein the pharmaceutical degradable polymer comprises any one or a combination of at least two of PLGA, PEG, mPEG-PLGA or DSPE-PEG;
optionally, a dosage form of the preparation comprises an oral dosage form, an injection dosage form or a topical dosage form;
wherein a mode of administration of the drug comprises digestive tract administration, injection administration, transdermal administration, mucosal administration, inhalation administration or topical administration;
wherein the combined composition further comprises a pharmaceutically acceptable adjuvant; and
wherein the adjuvant comprises any one or a combination of at least two of a carrier, a diluent, an adhesive, a wetting agent, a disintegrant, an emulsifier, a co-solvent, a solubilizer, an osmotic pressure regulator, a surfactant, a coating material, a colorant, a pH adjusting agent, an antioxidant, a bacteriostatic agent or a buffering agent.

7. The method according to claim 6, wherein the carrier-free nanoparticles are prepared through a preparation method comprising the following steps:
(1) dissolving celastrol or a pharmaceutical salt thereof and a hydrophobic antibiotic in a good solvent to obtain a solution A;
(2) adding the solution A to a poor solvent of celastrol or the pharmaceutical salt thereof and the hydrophobic antibiotic to obtain a solution B; and
(3) stirring the solution B, and separating free drugs to obtain the carrier-free nanoparticles;
optionally, the good solvent comprises any one or a combination of at least two of methanol, ethanol, propanol, isopropanol, acetone, tetrahydrofuran or dimethylsulfoxide;
wherein the poor solvent comprises deionized water and/or a phosphate buffer;
wherein the stirring is performed for 5-60 min; and
wherein ultrafiltration centrifugation is used in the separation of the free drugs, and conditions of the ultrafiltration centrifugation are a rotational speed of 8000-10000 g and a duration of 10-30 min.

8. The method according to claim 6, wherein the composite liposome is prepared through a preparation method comprising the following steps:
(1) mixing a first solvent, celastrol or a pharmaceutical salt thereof, a hydrophobic antibiotic, a phospholipid and cholesterol to obtain a solution A, and mixing a second solvent with a hydrophilic antibiotic to obtain a solution B;
(2) removing the first solvent in the solution A, mixing the solute with the solution B, and hydrating to obtain a solution C; and
(3) performing ultrasonication and gradient extrusion on the solution C to obtain the composite liposome;
wherein the first solvent comprises any one or a combination of at least two of methanol, ethanol, propanol or chloroform, and the second solvent comprises deionized water and/or a phosphate buffer;
wherein the hydration is performed for 10-60 min at a temperature of 15-35° C.;
wherein apertures of filter membranes for the gradient extrusion are 400 nm and 200 nm in sequence; and
wherein the ultrasonic treatment in step (3) is performed at 0-10° C., and the ultrasonication is performed at a power of 80-250 W for 3-30 min.

9. The method according to claim 6, wherein the composite liposome is prepared through a preparation method comprising the following steps:
(1) mixing a first solvent, a phospholipid and cholesterol to obtain a solution A, mixing celastrol or a pharmaceutical salt thereof, an antibiotic and an organic solvent, and adding the mixture dropwise to a second solvent to obtain a solution B;
(2) removing the first solvent in the solution A, mixing the solute with the solution B, and hydrating the solute to obtain a solution C; and
(3) performing ultrasonication and gradient extrusion on the solution C to obtain the composite liposome;
wherein the first solvent comprises any one or a combination of at least two of methanol, ethanol, propanol or chloroform, the organic solvent comprises any one or a combination of at least two of methanol, ethanol, dimethylformamide (DMF) or dimethyl sulfoxide (DMSO), and the second solvent comprises deionized water and/or a phosphate buffer;
wherein the hydration is performed for 10-30 min and a temperature of 15-35° C.;
wherein the ultrasonic treatment in step (3) is performed at 0-10° C., and the ultrasonication is performed at a power of 80-250 W for 3-30 min; and
wherein apertures of filter membranes for the gradient extrusion are 400 nm and 200 nm in sequence.

10. The method according to claim 6, wherein the composite polymer nanoparticles are prepared through a preparation method comprising the following steps:
(1) mixing a first solvent with a pharmaceutical degradable polymer to obtain a solution A, mixing celastrol or a pharmaceutical salt thereof, an antibiotic and an organic solvent, and adding the mixture dropwise to a second solvent containing an emulsifier to obtain a solution B;
(2) mixing the solution A with the solution B to obtain a solution C;
(3) adding the solution C to a third solvent containing an emulsifier, and mixing to obtain a solution D; and
(4) removing the first solvent remaining in the solution D to obtain the composite polymer nanoparticles;
wherein the first solvent comprises any one or a combination of at least two of methanol, ethanol, propanol or dichloromethane, the organic solvent comprises any one or a combination of at least two of methanol, ethanol, DMF or DMSO, the second solvent comprises deionized water and/or a phosphate buffer, and the third solvent comprises deionized water and/or a phosphate buffer;
wherein the emulsifiers in the second solvent and the third solvent each independently comprise any one or a combination of at least two of polyvinyl alcohol, polysorbate 80 or vitamin E polyethylene glycol succinate; and
wherein the emulsifier in the second solvent has a mass fraction of 3% to 15%, and the emulsifier in the third solvent has a mass fraction of 30% to 60%.

11. A combined pharmaceutical composition for resisting fungal and bacterial infections, wherein active components of the combined pharmaceutical composition are an antibiotic and an antibiotic adjuvant;
the antibiotic adjuvant is celastrol or a pharmaceutical salt thereof;
the antibiotic comprises any one or a combination of at least two of amphotericin B, nystatin, miconazole, ketoconazole, fluconazole, itraconazole, voriconazole, posaconazole, flucytosine, terbinafine, caspofungin, micafungin, anidulafungin, griseofulvin, penicillin, amoxicillin, ampicillin, cefradine, cefalexin, ceftazidime, imipenem, amikacin, gentamicin, doxycycline, tetracycline, minocycline, erythromycin, clarithromycin, azithromycin, sulfadiazine, ciprofloxacin, levofloxacin, metronidazole, clindamycin or fosfomycin;
wherein the fungi comprise any one or a combination of at least two of Candida albicans, Candida glabrata, Candida tropicalis, Candida krusei, Aspergillus fumigatus, Aspergillus niger, Aspergillus flavus, Aspergillus oryzae, Fusarium, Cryptococcus neoformans, Candida, Mucor, Histoplasma capsulatum, Scedosporium, Coccidioides, Cryptococcus gattii or Paracoccidioides; and
optionally, the bacteria comprise any one or a combination of at least two of Klebsiella pneumoniae, Acinetobacter baumannii, Pseudomonas aeruginosa, Escherichia coli, Salmonella, Shigella, Streptococcus pneumoniae, Staphylococcus aureus, Mycobacterium tuberculosis or Clostridioides difficile.

12. The combined pharmaceutical composition according to claim 11, wherein a molar ratio of the antibiotic to the antibiotic adjuvant is 1:(0.001-500).
